SEQUENCE LISTING (15 sequences)

SEQ ID NO: 1 - protein, 9 aa, Artificial sequence
  Synthetic amino acid
ASGPPAKAK

SEQ ID NO: 2 - protein, 10 aa, Artificial sequence
  Synthetic amino acid
SSQPSPSGPK

SEQ ID NO: 3 - protein, 10 aa, Artificial sequence
  Synthetic amino acid
SSQPSPSDPK

SEQ ID NO: 4 - protein, 26 aa, Artificial sequence
  Synthetic amino acid
ASGPPAKAKETGKRKSSSQPSPSGPK

SEQ ID NO: 5 - protein, 26 aa, Artificial sequence
  Synthetic amino acid
ASGPPAKAKETGKRKSSSQPSPSDPK

SEQ ID NO: 6 - protein, 32 aa, Artificial sequence
  Synthetic amino acid
MSPKPRASGPPAKAKETGKRKSSSQPSPSGPK

SEQ ID NO: 7 - protein, 32 aa, Artificial sequence
  Synthetic amino acid
MSPKPRASGPPAKAKETGKRKSSSQPSPSDPK

SEQ ID NO: 8 - protein, 9 aa, Artificial sequence
  Synthetic amino acid
RASGPPAKA

SEQ ID NO: 9 - protein, 9 aa, Artificial sequence
  Synthetic amino acid
AKAKETGKR

SEQ ID NO: 10 - protein, 9 aa, Artificial sequence
  Synthetic amino acid
KGAATKMAA

SEQ ID NO: 11 - protein, 9 aa, Artificial sequence
  Synthetic amino acid
GAATKMAAV

SEQ ID NO: 12 - protein, 15 aa, Artificial sequence
  Synthetic amino acid
MSPKPRASGPPAKAK

SEQ ID NO: 13 - protein, 15 aa, Artificial sequence
  Synthetic amino acid
SGPPAKAKETGKRKS

SEQ ID NO: 14 - protein, 15 aa, Artificial sequence
  Synthetic amino acid
PAKAKETGKRKSSSQ

SEQ ID NO: 15 - protein, 15 aa, Artificial sequence
  Synthetic amino acid
GRRGKKGAATKMAAV

(19) United States
(12) Patent Application Publication
(10) Pub. No.: US 2022/0372092 A1
(43) Pub. Date: Nov. 24, 2022

(54) HLA-RESTRICTED VCX/Y PEPTIDES AND T CELL RECEPTORS AND USE THEREOF

(71) Applicant: Board of Regents, The University of Texas System, Austin, TX (US)

(72) Inventors: Cassian YEE, Houston, TX (US); Ke PAN, Houston, TX (US)

(73) Assignee: Board of Regents, The University of Texas System, Austin, TX (US)

(21) Appl. No.: 17/769,839

(22) PCT Filed: Oct. 16, 2020
(86) PCT No.: PCT/US2020/056049

§ 371 Date: Apr. 18, 2022

Related U.S. Application Data
(60) Provisional application No. 62/923,228, filed on Oct. 18, 2019.

(51) Int. Cl. C07K 14/47; C07K 14/74; A61K 35/17; A61K 39/00; A61K 39/395; C12N 5/0783; C07K 14/725

(52) U.S. Cl. CPC C07K 14/4748; C07K 14/70539; A61K 35/17; A61K 39/001184; A61K 39/3955; C12N 5/0638; C07K 14/7051; C12N 2502/1121

(57) ABSTRACT

Provided herein are tumor-antigen VCX/Y specific peptides. Also provided herein are methods of generating VCX/Y-specific immune cells and their use for the treatment of cancer. Immunogenic compositions comprising VCX/Y-specific peptides are also provided. In a further embodiment, there is provided a protein complex comprising a peptide according to any of the embodiments and aspects described above in complex with HLA. In some aspects, the HLA is a HLA-A11, HLA-DR, or HLA-DQ.

This application claims the benefit of U.S. Provisional Patent Application Ser. No. 62/923,228, filed Oct. 18, 2019, the entirety of which is incorporated herein by reference.

### INCORPORATION OF SEQUENCE LISTING

The sequence listing that is contained in the file named “UTSCP1475WO_ST25.txt”, which is 5 KB (as measured in Microsoft Windows®) and was created on Oct. 13, 2020, is filed herewith by electronic submission and is incorporated by reference herein.

### BACKGROUND OF THE INVENTION

### 1. Field of the Invention

The present invention relates generally to the fields of immunology and medicine. More particularly, it concerns tumor antigen peptides and uses thereof for the treatment of cancer.

### 2. Description of Related Art

T cell based therapies have shown significant promise as a method for treating many cancers; unfortunately, this approach has also been hindered by a paucity of immunogenic antigen targets for common cancers and potential toxicity to non-cancerous tissues. These T cell based therapies can include ACT (adoptive cell transfer) and vaccination approaches. ACT generally involves which involves infusing a large number of autologous activated tumor-specific T cells into a patient, e.g., to treat a cancer. ACT has resulted in therapeutic clinical responses in melanoma patients (Yee 2002; Dudley 2002; Yee 2014). Generally, to develop effective anti-tumor T cell responses, the following three steps are normally required: priming and activating antigen-specific T cells, migrating activated T cells to tumor site, and recognizing and killing tumor by antigen-specific T cells. The choice of target antigen is important for induction of effective antigen-specific T cells.

While several tumor-associated antigens have been identified for melanoma and a handful of other solid tumor malignancies, there are few immunogenic targets for pancreatic, ovarian, gastric, lung, cervical, breast, and head and neck cancer. Identification and validation of novel epitopes and target antigens for these common and difficult to treat malignancies is warranted.

### SUMMARY OF THE INVENTION

In a first embodiment, the present disclosure provides an isolated VCX/Y peptide comprising (i) the amino acid sequence of SEQ ID NO: 1 (ASGPPAKAK), SEQ ID NO: 2 (SSQPSPSGPK), SEQ ID NO: 3 (SSQPSPSDPK), SEQ ID NO: 8 (RASGPPAKA), SEQ ID NO: 9 (AKAKETGKR), SEQ ID NO: 10 (KGAATKMAA), or SEQ ID NO: 11 (GAATKMAAV), or (ii) an amino acid sequence having no more than 1 amino acid substitution or deletion relative to SEQ ID NO:1, SEQ ID NO: 2, SEQ ID NO: 3, SEQ ID NO: 8, SEQ ID NO: 9, SEQ ID NO: 10, or SEQ ID NO: 11 wherein the peptide induces cytotoxic T lymphocytes (CTLs) and selectively binds to HLA. In one embodiment, there is provided an isolated VCX/Y peptide of 35 amino acids in length or less comprising (i) the amino acid sequence of SEQ ID NO: 1 (ASGPPAKAK), SEQ ID NO: 2 (SSQPSPSGPK), SEQ ID NO: 3 (SSQPSPSDPK), SEQ ID NO: 8 (RASGPPAKA), SEQ ID NO: 9 (AKAKETGKR), SEQ ID NO: 10 (KGAATKMAA), or SEQ ID NO: 11 (GAATKMAAV), or (ii) an amino acid sequence having no more than 1 amino acid substitution or deletion relative to SEQ ID NO:1, SEQ ID NO: 2, SEQ ID NO: 3, SEQ ID NO: 8, SEQ ID NO: 9, SEQ ID NO: 10, or SEQ ID NO: 11 wherein the peptide is capable of inducing cytotoxic T lymphocytes (CTLs) and selectively binds to HLA. In certain aspects, the peptide comprises (i) SEQ ID NO: 1 and SEQ ID NO: 2 or (ii) SEQ ID NO: 1 and SEQ ID NO: 3 or (iii) an amino acid sequence having no more than 1 amino acid substitution or deletion relative to SEQ ID NO: 1 and an amino acid sequence having no more than 1 amino acid substitution or deletion relative to SEQ ID NO: 2 or (iv) an amino acid sequence having no more than 1 amino acid substitution or deletion relative to SEQ ID NO: 1 and an amino acid sequence having no more than 1 amino acid substitution or deletion relative to SEQ ID NO: 3. In a further aspect, the peptide is capable of inducing cytotoxic T lymphocytes (CTLs) and selectively binds to HLA-A11. In specific aspects, the peptide is 32 amino acids or less in length and comprises the amino acid sequence of SEQ ID NO: 1 and SEQ ID NO: 2. In some aspects, the peptide comprises the amino acid sequence of SEQ ID NO: 2. In specific aspects, the peptide is 32 amino acids or less in length and comprises the amino acid sequence of SEQ ID NO: 1 and SEQ ID NO: 3. In other aspects, the peptide comprises the amino acid sequence of SEQ ID NO: 4 (ASGPPAKAKETGKRKSSSQPSPSGPK) or SEQ ID NO: 5 (ASGPPAKAKETGKRKSSSQPSPSDPK). In additional aspects, the peptide comprises the amino acid sequence of SEQ ID NO: 6 (MSPKPRASGPPAKAKETGKRKSSSQPSPSGPK) or SEQ ID NO: 7 (MSPKPRASGPPAKAKETGKRKSSSQPSPSDPK). In some aspects, the peptide comprises an amino acid sequence at least 90% or 95% identical to any one of SEQ ID NOs: 4-7.

In further aspects, the peptide is 32 amino acids or less in length and comprises the amino acid sequence of SEQ ID NO: 8, SEQ ID NO: 9, SEQ ID NO: 10, SEQ ID NO: 11 or an amino acid sequence having no more than 1 amino acid substitution or deletion relative to SEQ ID NO: 8, SEQ ID NO: 9, SEQ ID NO: 10, or SEQ ID NO: 11. In certain aspects, the peptide is capable of inducing cytotoxic T lymphocytes (CTLs) and selectively binds to HLA-DR or HLA-DQ. In a particular aspect, the peptide is 26 amino acids or less in length and comprises the amino acid sequence of SEQ ID NO: 8. In some specific aspects, the peptide comprises the amino acid sequence of SEQ ID NO: 12 or an amino acid sequence at least 90% identical to SEQ ID NO: 12. In another aspect, the peptide is 26 amino acids or less in length and comprises the amino acid sequence of SEQ ID NO: 9. In several aspects, the peptide comprises the amino acid sequence of SEQ ID NO: 13-14 or an amino acid sequence at least 90% identical to SEQ ID NO: 13-14. In other aspects, the peptide is 26 amino acids or less in length and comprises the amino acid sequence of SEQ ID NO: 10. In certain particular aspects, the peptide comprises the amino acid sequence of SEQ ID NO: 15 or an amino acid sequence at least 90% identical to SEQ ID NO: 15. In some aspects, the peptide is 26 amino acids or less in length and comprises the amino acid sequence of SEQ ID NO: 11. In specific aspects, the peptide comprises the amino acid sequence of SEQ ID NO: 15 or an amino acid sequence at least 90% identical to SEQ ID NO: 15.

In still further aspects, the peptide is 15, 16, 17, 18, 19, 20, 21, 22, 23, 24, 25, 26, 27, 28, 29, or 30 amino acids in length or less. In certain aspects, the peptide consists of any one of SEQ ID NOs: 1-15.

In a further embodiment, there is provided a protein complex comprising a peptide according to any of the embodiments and aspects described above in complex with HLA. In some aspects, the HLA is a HLA-A11, HLA-DR, or HLA-DQ.

In yet a further embodiment, the invention provides a pharmaceutical composition comprising the peptide of any of the embodiments and aspects described above and a pharmaceutical carrier. In certain aspects, the pharmaceutical carrier is a buffer salt solution. In some aspects, the pharmaceutical carrier is a saline solution, phosphate buffered saline (PBS) or Ringer's lactate. In particular aspects, the pharmaceutical composition is formulated for parenteral administration, intravenous injection, intramuscular injection, inhalation, or subcutaneous injection. In certain aspects, the peptide is complexed with or is contained within a liposome, lipid-containing nanoparticle, or in a lipid-based carrier. In some specific aspects, the pharmaceutical preparation is formulated for injection or inhalation as a nasal spray. In another aspect, the pharmaceutical preparation further comprises an adjuvant component.

In still yet a further embodiment, there is provided an isolated nucleic acid encoding the peptide of any of the embodiments and aspects described above. Yet another embodiment provides a vector comprising a contiguous sequence consisting of said nucleic acid encoding the peptide of any of the embodiments and aspects described above. And yet a further embodiment of the invention provides a host cell comprising the peptide of any of the embodiments and aspects described above or a vector comprising a contiguous sequence consisting of said nucleic acid encoding the peptide of any of the embodiments and aspects described above. In some aspects, the cell is not a cancer cell. In other aspects, the cell is an antigen presenting cell.

A further embodiment of the invention provides a method of promoting an immune response in a subject, comprising administering an effective amount of the peptide of any of the embodiments and aspects described above to the subject, wherein the peptide induces VCX/Y-specific T cells in the subject. In certain aspects, a peptide of the embodiments is administered in conjunction with at least a first adjuvant. In some aspects, the peptide (or mixture of peptides) is administered in conjunction with TLR ligand. In some aspects, the TLR ligand is a TLR2, TLR3, TLR4, TLR7, TLR8 or TLR9 agonist. In some aspects, the TLR ligand is a TLR7 agonist. In some aspects, the TLR7 agonist is CL075, CL097, CL264, CL307, GS-9620, Poly(dT), imiquimod, gardiquimod, resiquimod (R848), loxoribine or a ssRNA oligonucleotide. In some aspects, the TLR7 agonist is imiquimod.

In certain aspects, a subject for treatment according to the embodiments is diagnosed with cancer. In some specific aspects, the cancer is testicular cancer, thymoma, bladder cancer, uterine carcinoma, melanoma, sarcoma, cervix cancer, or head and neck cancer. In a particular aspect, the subject is a human. In additional aspects, the method further comprises administering at least a second anti-cancer therapy. In several aspects, the second anti-cancer therapy is selected from the group consisting of a chemotherapy, a radiotherapy, an immunotherapy, or a surgery. In a specific aspect, the immunotherapy comprises at least one immune checkpoint inhibitor. In a further aspect, the immune checkpoint inhibitor is an anti-PD1, anti-PDL1 or anti-CTLA-4 monoclonal antibody. In other aspects, the immunotherapy is a combination of immune checkpoint inhibitors. In particular aspects, the combination of immune checkpoint inhibitors is anti-PD1 and anti-CTLA-4 monoclonal antibodies. In some aspects, the combination of immune checkpoint inhibitors is anti-PDL1 and anti-CTLA-4 monoclonal antibodies. In certain aspects, the PD-1 monoclonal antibody is selected from the group consisting of nivolumab, pembrolizumab and cemiplimab. In particular aspects, the PD-L1 monoclonal antibody is selected from the group consisting of atezolizumab, avelumab and durvalumab. In specific aspects, the CTLA-4 monoclonal antibody is ipilimumab.

In still yet a further embodiment, there is provided a method of producing VCX/Y-specific immune effector cells comprising: (a) obtaining a starting population of immune effector cells; and (b) contacting the starting population of immune effector cells with the VCX/Y peptide of any of the embodiments and aspects described above, thereby generating VCX/Y-specific immune effector cells. In some aspects, contacting is further defined as co-culturing the starting population of immune effector cells with antigen presenting cells (APCs), wherein the APCs present the VCX/Y peptide of the first embodiment on their surface. In another aspect, the APCs are dendritic cells. In certain aspects, the immune effector cells are T cells, peripheral blood lymphocytes, NK cells, invariant NK cells, NKT cells. In several aspects, the immune effector cells have been differentiated from mesenchymal stem cell (MSC) or induced pluripotent stem (iPS) cells. In a further aspect, the T cells are CD8⁺ T cells, CD4⁺ T cells, or γδ T cells. In other aspects, the T cells are cytotoxic T lymphocytes (CTLs). In certain specific aspects, obtaining comprises isolating the starting population of immune effector cells from peripheral blood mononuclear cells (PBMCs). In some aspects, the starting population of immune effector cells is obtained from a subject. In particular aspects, the subject is a human. In certain aspects, the subject has cancer.

In additional methods, the method further comprises introducing the VCX/Y peptides or a nucleic acid encoding the VCX/Y peptide into the dendritic cells prior to the co-culturing. In some aspects, the peptide or nucleic acids encoding the peptide are introduced by electroporation. In certain aspects, the peptide or nucleic acids encoding the peptide are introduced by adding the peptides or nucleic acids encoding the peptides to the dendritic cell culture media. In some aspects, the immune effector cells are co-cultured with a second population of dendritic cells into which the peptide or the nucleic acid encoding the peptide has been introduced. In certain aspects, a population of CD8-positive and VCX/Y peptide MEW tetramer-positive T cells are purified from the immune effector cells following the co-culturing. In particular aspects, a clonal population of VCX/Y-specific immune effector cells are generated by limiting or serial dilution followed by expansion of individual clones by a rapid expansion protocol. In some aspects, the method further comprises cloning of a T cell receptor (TCR) from the clonal population of VCX/Y-specific immune effector cells. In certain aspects, cloning of the TCR is cloning of a TCR alpha and a beta chain. In some aspects, the TCR is cloned using a 5′-Rapid amplification of cDNA ends (RACE) method. In certain aspects, the cloned TCR is subcloned into an expression vector. In some aspects, the expression vector is a retroviral or lentiviral vector. In specific aspects, a host cell is transduced with the expression vector to generate an engineered cell that expresses the TCR. For example, the host cell is an immune cell, such as a T cell and the engineered cell is an engineered T cell. In particular aspects, the T cell is a CD8⁺ T cell, CD4⁺ T cell, or γδ T cell and the engineered cell is an engineered T cell. In some aspects, the starting population of immune effector cells is obtained from a subject with cancer and the host cell is allogeneic or autologous to the subject. In certain aspects, a population of CD8-positive and VCX/Y peptide MEW tetramer-positive engineered T cells are purified from the transduced host cells. In particular aspects, a clonal population of VCX/Y-specific engineered T cells are generated by limiting or serial dilution followed by expansion of individual clones by a rapid expansion protocol.

Further provided herein is a VCX/Y-specific engineered T cell produced according to any one of the methods of any of the embodiments and aspects, described above. Yet a further embodiment of the invention provides a VCX/Y-specific T cell produced according to any of the embodiments and aspects described above. In still a further embodiment, there is provided a pharmaceutical composition comprising the VCX/Y-specific T cells produced according to any one of the embodiments and aspects described above. And yet a further embodiment provides a composition comprising an effective amount of the VCX/Y-specific T cells produced according to any of the embodiments and aspects described above for the treatment of cancer in a subject.

Still yet a further embodiment of the invention provides a method of treating cancer in a subject comprising administering an effective amount of the VCX/Y-specific T cells produced according to any of the embodiments and aspects described above to the subject. In some aspects, the cancer is thymoma, bladder cancer, uterine carcinoma, melanoma, sarcoma, cervix cancer, or head and neck cancer. In specific aspects, the subject is a human. In certain aspects, the VCX/Y-specific T cells are autologous or allogeneic. In further aspects, the method additionally comprises lymphodepletion of the subject prior to administration of the VCXIY-specific T cells. In particular aspects, lymphodepletion comprises administration of cyclophosphamide and/or fludarabine. In additional aspects, the method further comprises administering at least a second therapeutic agent. In some specific aspects, the at least a second therapeutic agent comprises chemotherapy, immunotherapy, surgery, radiotherapy, or biotherapy. In particular aspects, the immunotherapy comprises at least one immune checkpoint inhibitor. In further aspects, the immune checkpoint inhibitor is an anti-PD1, anti-PDL1, or anti-CTLA-4 monoclonal antibody. In another aspect, the immunotherapy is a combination of immune checkpoint inhibitors. In certain specific aspects, the combination of immune checkpoint inhibitors is anti-PD1 and anti-CTLA-4 monoclonal antibodies. In certain aspects, the PD-1 monoclonal antibody is selected from the group consisting of nivolumab, pembrolizumab and cemiplimab. In particular aspects, the PD-L1 monoclonal antibody is selected from the group consisting of atezolizumab, avelumab and durvalumab. In specific aspects, the CTLA-4 monoclonal antibody is ipilimumab. In some aspects, the VCX/Y-specific T cells and/or the at least a second therapeutic agent are administered intravenously, intraperitoneally, intratracheally, intratumorally, intramuscularly, endoscopically, intralesionally, percutaneously, subcutaneously, regionally, or by direct injection or perfusion. In further aspects, the subject is determined to have cancer cells which express a protein of the VCX/Y family. In certain aspects, the protein is VCX3B or VCY.

A further embodiment provides a method of cloning a VCX/Y T cell receptor (TCR), the method comprising (a) obtaining a starting population of immune effector cells; (b) contacting the starting population of immune effector cells with the VCX/Y peptide of any of the present embodiments or aspects thereof, thereby generating VCX/Y-specific immune effector cells; (c) purifying immune effector cells specific to the VCX/Y peptide, (d) isolating a TCR sequence from the purified immune effector cells.

In some aspects, contacting is further defined as co-culturing the starting population of immune effector cells with antigen presenting cells (APCs), wherein the APCs present the VCX/Y peptide of any of the present embodiments or aspects thereof on their surface. the APCs are dendritic cells. In certain aspects, the immune effector cells are T cells, peripheral blood lymphocytes, NK cells, invariant NK cells, NKT cells. In particular aspects, the immune effector cells have been differentiated from mesenchymal stem cell (MSC) or induced pluripotent stem (iPS) cells. In some aspects, the T cells are CD8⁺ T cells, CD4⁺ T cells, or γδ T cells. In specific aspects, the T cells are cytotoxic T lymphocytes (CTLs). In some aspects, obtaining comprises isolating the starting population of immune effector cells from peripheral blood mononuclear cells (PBMCs). In certain aspects, the starting population of immune effector cells is obtained from a subject. In some aspects, the subject is a human. In specific aspects, the subject has cancer.

In additional aspects, the method further comprises introducing the VCX/Y peptide or a nucleic acid encoding the VCX/Y peptide into the dendritic cells prior to the co-culturing. In some aspects, the peptide or nucleic acid encoding the peptide are introduced by electroporation. In certain aspects, the peptide or nucleic acid encoding the peptide are introduced by adding the peptides or nucleic acid encoding the peptides to the media of the dendritic cells. In some aspects, the immune effector cells are co-cultured with a second population of dendritic cells into which the VCX/Y peptide or a nucleic acid encoding the VCX/Y peptide has been introduced.

In particular aspects, purifying is defined as purifying a population of CD8-positive and VCX/Y peptide MEW tetramer-positive T cells from the immune effector cells following the co-culturing. In some aspects, the population of CD8-positive and VCX/Y peptide MEW tetramer-positive T cells are purified by fluorescence activated cell sorting (FACS). In certain aspects, purifying further comprises generation of a clonal population of VCX/Y-specific immune effector cells by limiting or serial dilution of sorted cells followed by expansion of individual clones by a rapid expansion protocol.

In some aspects, isolating is defined as cloning of a T cell receptor (TCR) from the clonal population of VCX/Y-specific immune effector cells. In particular aspects, cloning of the TCR is cloning of a TCR alpha and a beta chain. In some aspects, the TCR alpha and beta chains are cloned using a 5′-Rapid amplification of cDNA ends (RACE) method. In some aspects, the cloned TCR is subcloned into an expression vector. In certain aspects, the expression vector comprises a linker domain between the TCR alpha sequence and TCR beta sequence. In some aspects, the linker domain comprises a sequence encoding one or more peptide cleavage sites. In particular aspects, the one or more cleavage sites are a Furin cleavage site and/or a P2A cleavage site. In specific aspects, the one or more cleavage sites are separated by a spacer, such as SGSG or GSG. In some aspects, the TCR alpha sequence and TCR beta sequence are linked by an IRES sequence. In certain aspects, the expression vector is a retroviral or lentiviral vector. In particular aspects, a host cell is transduced with the expression vector to generate an engineered cell that expresses the TCR alpha and beta chains. In some aspects, the host cell is an immune cell, such as a T cell and the engineered cell is an engineered T cell. In certain aspects, the T cell is a CD8⁺ T cell, CD4⁺ T cell, or γδ T cell and the engineered cell is an engineered T cell. In some aspects, a population of CD8-positive and VCX/Y peptide MEW tetramer-positive engineered T cells are purified from the transduced host cells. In particular aspects, a clonal population of VCX/Y-specific engineered T cells are generated by limiting or serial dilution after purification followed by expansion of individual engineered T cell clones by a rapid expansion protocol.

Another embodiment provides a VCX/Y-specific engineered T cell expressing a TCR produced according to any one of the present embodiments or aspects thereof.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: A schematic of the experimental procedure for MEW peptide identification.

FIG. 2: Figure shows FACS histograms of K562-A11-VCY cell line generation.

FIG. 3: Graph show mass spectrometry analysis of VCY HLA-A1101 restricted peptide identification from mild acid direct elution.

FIG. 4: Graph show mass spectrometry analysis of VCY HLA-A1101 restricted peptide identification from MEW immunoaffinity chromatography method.

FIG. 5: Graph show mass spectrometry analysis of VCY HLA-A1101 restricted peptide identification from MEW immunoaffinity chromatography method.

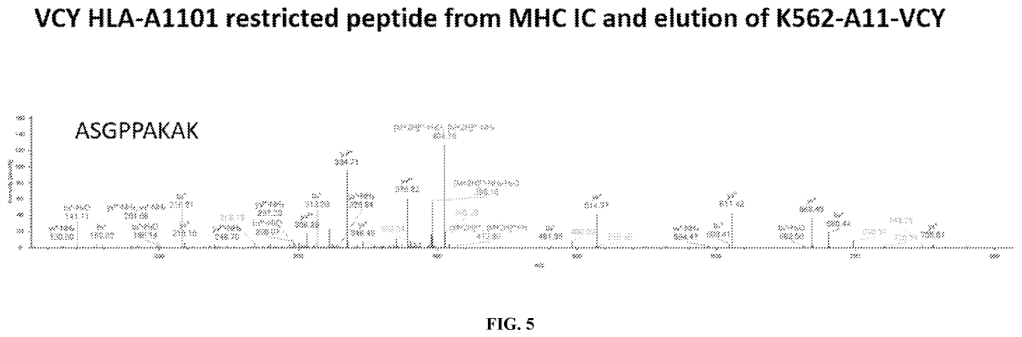

### DESCRIPTION OF ILLUSTRATIVE EMBODIMENTS

For patients with many different cancer types, immune effector cell-based immunotherapies represent a promising approach with proven efficacy. However, antigen-specific cell-based therapies for most cancer types are not feasible due to the lack of tumor-associated antigens currently known, which has stalled their clinical development. Studies detailed herein identified novel VCX/Y family derived HLA-A11 restricted peptide epitopes found in all of the VCX/Y family members. Using the peptide epitopes, additional epitopes with predicted specificity for more prevalent HLA classes, such as HLA-DR and HLA-DQ were also identified. The provided peptide epitopes can be use, for example to formulate immunogenic composition to elicit an anti-tumor antigen immune response in treated subjects. Likewise, the peptides (e.g., in complex with an appropriate HLA complex or expressed in antigen presenting cells) can be used to stimulate growth of tumor-specific immune effector cells, such as T-cells ex vivo. Such produced antigen-specific CTLs may be used to target solid cancers (e.g., pancreatic, testicular, ovarian, gastric, and breast cancers).

Accordingly, the present disclosure provides tumor antigen-specific peptides, such as to tumor antigen VCX/Y, for the use as immunotherapy for the treatment of a cancer. An exemplary VCX/Y peptide, include those provided as SEQ ID NOs: 1-15. For example, a tumor antigen-specific peptide may be contacted with or used to stimulate a population of T cells to induce proliferation of the T cells that recognize or bind the tumor antigen-specific peptide. In other embodiments, a VCXIY-specific peptide of the present disclosure may be administered to a subject, such as a human patient, to enhance the immune response of the subject against a cancer.

A VCX/Y-specific peptide may be included in an active immunotherapy (e.g., a cancer vaccine) or a passive immunotherapy (e.g., an adoptive immunotherapy). Active immunotherapies include immunizing a subject with a purified tumor antigen or an immunodominant VCX/Y-specific peptide (native or modified); alternately, antigen presenting cells pulsed with a VCX/Y-specific peptide (or transfected with genes encoding the tumor antigen) may be administered to a subject. The VCX/Y-specific peptide may be modified or contain one or more mutations such as, e.g., a substitution mutation. Passive immunotherapies include adoptive immunotherapies. Adoptive immunotherapies generally involve administering cells to a subject, wherein the cells (e.g., cytotoxic T cells) have been sensitized in vitro to the VCX/Y-specific peptide (see, e.g., U.S. Pat. No. 7,910,109).

In particular, a patient's own VCX/Y-specific T cells can be generated ex vivo for effective immune-based therapies within a short period of time, such as 6 to 8 weeks. The T cells may be isolated and expanded from autologous or allogeneic T cells (e.g., CD4⁺ T cells, CD8⁺ T cells, γδ T cells and Tregs) isolated from peripheral blood, such as with the tetramer guided sorting and rapid expansion protocol (REP). Next, the peptide or corresponding coded polynucleotides can be loaded to HLA (e.g., HLA-A11, HLA-DR or HLA-DQ) positive dendritic cells, LCL, PBMC, or artificial antigen presenting cells (aAPCs), and then co-cultured with the T cells by several rounds of stimulation to generate antigen-specific CTL cell lines or clones. Furthermore, with manipulation of immune modulating parameters, the effector function and long-term persistence in vivo of these expanded antigen specific T cells can be enhanced. These autologous CTL cells can be used for adoptive immunotherapy for VCX/Y positive cancer patients, who express the appropriate HLA subtype for the given peptide. Further, other VCX/Y-specific cells that can be generated from the present disclosure include autologous or allogeneic NK cells, invariant NK cells, NKT cells, mesenchymal stem cells (MSCs), and induced pluripotent stem (iPS) cells. These cells may be isolated from blood or the umbilical cord.

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “essentially” is to be understood that methods or compositions include only the specified steps or materials and those that do not materially affect the basic and novel characteristics of those methods and compositions.

The term “substantially free of” is used to 98% of the listed components and less than 2% of the components to which composition or particle is substantially free of.

The terms “substantially” or “approximately” as used herein may be applied to modify any quantitative comparison, value, measurement, or other representation that could permissibly vary without resulting in a change in the basic function to which it is related.

The term “about” means, in general, within a standard deviation of the stated value as determined using a standard analytical technique for measuring the stated value. The terms can also be used by referring to plus or minus 5% of the stated value.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a T cell therapy.

“Subject” and “patient” refer to either a human or non-human, such as primates, mammals, and vertebrates. In particular embodiments, the subject is a human.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

An “anti-cancer” agent is capable of negatively affecting a cancer cell/tumor in a subject, for example, by promoting killing of cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity, such as an ScFv (single chain fragment variable) antibody, Fab fragment, F(ab′)2 fragment, or Fv fragment. For example, a monoclonal antibody may be of the IgG1, IgG2, IgG3 or IgG4 type.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “unit dose” or “dosage” refers to physically discrete units suitable for use in a subject, each unit containing a predetermined quantity of the therapeutic composition calculated to produce the desired responses discussed above in association with its administration, i.e., the appropriate route and treatment regimen. The quantity to be administered, both according to number of treatments and unit dose, depends on the effect desired. The actual dosage amount of a composition of the present embodiments administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 μg/kg/body weight to about 1000 mg/kg/body weight (this such range includes intervening doses) or more per administration, and any range derivable therein. In non-limiting examples of a derivable range from the numbers listed herein, a range of about 5 μg/kg/body weight to about 100 mg/kg/body weight, about 5 μg/kg/body weight to about 500 mg/kg/body weight, etc., can be administered. The practitioner responsible for administration will, in any event, determine the concentration of active ingredient(s) in a composition and appropriate dose(s) for the individual subject. In some embodiments, the dosage of antigen-specific T cell infusion may comprise about 100 million to about 30 billion cells, such as 10, 15, or 20 billion cells.

The term “immune checkpoint” refers to a molecule such as a protein in the immune system which provides signals to its components in order to balance immune reactions. Known immune checkpoint proteins comprise CTLA-4, PD1 and its ligands PD-L1 and PD-L2 and in addition LAG-3, BTLA, B7H3, B7H4, TIM3, KIR. The pathways involving LAG3, BTLA, B7H3, B7H4, TIM3, and KIR are recognized in the art to constitute immune checkpoint pathways similar to the CTLA-4 and PD-1 dependent pathways (see e.g. Pardoll, 2012; Mellman et al., 2011.

An “immune checkpoint inhibitor” refers to any compound inhibiting the function of an immune checkpoint protein. Inhibition includes reduction of function and full blockade. In particular, the immune checkpoint protein is a human immune checkpoint protein. Thus, the immune checkpoint protein inhibitor in particular is an inhibitor of a human immune checkpoint protein.

As used herein, a “protective immune response” refers to a response by the immune system of a mammalian host to a cancer. A protective immune response may provide a therapeutic effect for the treatment of a cancer, e.g., decreasing tumor size or increasing survival.

As used herein, the term “antigen” is a molecule capable of being bound by an antibody or T-cell receptor. An antigen may generally be used to induce a humoral immune response and/or a cellular immune response leading to the production of B and/or T lymphocytes.

The terms “tumor-associated antigen,” “tumor antigen” and “cancer cell antigen” are used interchangeably herein. In each case, the terms refer to proteins, glycoproteins or carbohydrates that are specifically or preferentially expressed by cancer cells.

The term “chimeric antigen receptors (CARs),” as used herein, may refer to artificial T-cell receptors, chimeric T-cell receptors, or chimeric immunoreceptors, for example, and encompass engineered receptors that graft an artificial specificity onto a particular immune effector cell. CARs may be employed to impart the specificity of a monoclonal antibody onto a T cell, thereby allowing a large number of specific T cells to be generated, for example, for use in adoptive cell therapy. In specific embodiments, CARs direct specificity of the cell to a tumor associated antigen, for example. In some embodiments, CARs comprise an intracellular activation domain, a transmembrane domain, and an extracellular domain comprising a tumor associated antigen binding region. In particular aspects, CARs comprise fusions of single-chain variable fragments (scFv) derived from monoclonal antibodies, fused to CD3-zeta a transmembrane domain and endodomain. The specificity of other CAR designs may be derived from ligands of receptors (e.g., peptides) or from pattern-recognition receptors, such as Dectins. In certain cases, the spacing of the antigen-recognition domain can be modified to reduce activation-induced cell death. In certain cases, CARs comprise domains for additional co-stimulatory signaling, such as CD3ζ, FcR, CD27, CD28, CD137, DAP10, and/or OX40. In some cases, molecules can be co-expressed with the CAR, including co-stimulatory molecules, reporter genes for imaging (e.g., for positron emission tomography), gene products that conditionally ablate the T cells upon addition of a pro-drug, homing receptors, chemokines, chemokine receptors, cytokines, and cytokine receptors.

A polynucleotide or polynucleotide region (or a polypeptide or polypeptide region) has a certain percentage (for example, 80%, 85%, 90%, or 95%) of “sequence identity” or “homology” to another sequence means that, when aligned, that percentage of bases (or amino acids) are the same in comparing the two sequences. This alignment and the percent homology or sequence identity can be determined using software programs known in the art, for example those described in CURRENT PROTOCOLS IN MOLECULAR BIOLOGY (F. M. Ausubel et al., eds., 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR.

### II. VCX/Y PEPTIDES

Embodiments of the present disclosure concern tumor antigen-specific peptides, such as to the VCX/Y tumor antigen. In particular embodiments, the tumor antigen-specific peptides have the amino acid sequence of a VCX/Y peptide (SEQ ID NO: 1-15). In some aspects, the peptide is no more than 50, 45, 40, 35, 30, 25, 20 or 15 amino acids in length. In further aspects, the peptide of SEQ ID NO: 1-15 is fused to polypeptide having a non-VCX/Y amino acid sequence (i.e., a heterologous polypeptide). In some aspects the tumor antigen-specific peptide may have an amino acid sequence with at least 80, 85, 90, 95, 96, 97, 98, 99, or 100 percent sequence identity with the peptide sequence of SEQ ID NO:1-15 or a sequence according to SEQ ID NO: 1-15, but including 1, 2 or 3 amino acid substitutions or deletions relative to SEQ ID NO: 1-15.

As used herein, the term “peptide” encompasses amino acid chains comprising 7-35 amino acids, preferably 8-35 amino acid residues, and even more preferably 8-25 amino acids, or 7, 8, 9, 10, 11, 12, 13, 14, 15, 16, 17, 18, 19, 20, 21, 22, 23, 24, 25, 26, 27, 28, 29, 30, 31, 32, 33, 34, or 35 amino acids in length, or any range derivable therein. For example, a VCX/Y peptide of the present disclosure may, in some embodiments, comprise or consist of the sequence of any one of SEQ ID NOs: 1-15. As used herein, an “antigenic peptide” is a peptide which, when introduced into a vertebrate, can stimulate the production of antibodies in the vertebrate, i.e., is antigenic, and wherein the antibody can selectively recognize and/or bind the antigenic peptide. An antigenic peptide may comprise an immunoreactive VCX/Y peptide, and may comprise additional sequences. The additional sequences may be derived from a native antigen and may be heterologous, and such sequences may, but need not, be immunogenic. In some embodiments, a tumor antigen-specific peptide (e.g., a VCX/Y peptide) can selectively bind with HLA-A11, HLA-DB or HLA-DR. In certain embodiments, the VCX/Y peptide is 8, 9, 10, 11, 12, 13, 14, 15, 16, 17, 18, 19, 20, 21, 22, 23, 24, 25, 26, 27, 28, 29, 30, 31, 32, 33, 34, or 35 amino acids in length, or any range derivable therein. Preferably, the tumor antigen-specific peptide (e.g., a VCX/Y peptide) is from 8 to 35 amino acids in length. In some embodiments, the tumor antigen-specific peptide (e.g., a VCX/Y peptide) is from 8 to 10 amino acids in length.

As would be appreciated by one of skill in the art, MHC molecules can bind peptides of varying sizes, but typically not full-length proteins. While MHC class I molecules have been traditionally described to bind to peptides of 8-11 amino acids long, it has been shown that peptides 15 amino acids in length can bind to MEW class I molecules by bulging in the middle of the binding site or extending out of the MEW class I binding groove (Guo et al., 1992; Burrows et al., 2006; Samino et al., 2006; Stryhn et al., 2000; Collins et al., 1994; Blanchard and Shastri, 2008). Further, recent studies also demonstrated that longer peptides may be more efficiently endocytosed, processed, and presented by antigen-presenting cells (Zwaveling et al., 2002; Bijker et al., 2007; Melief and van der Burg, 2008; Quintarelli et al., 2011). As demonstrated in Zwaveling et al. (2002) peptides up to 35 amino acids in length may be used to selectively bind a class II MEW and are effective. As would be immediately appreciated by one of skill, a naturally occurring full-length tumor antigen, such as VCX/Y, would not be useful to selectively bind a class II MEW such that it would be endocytosed and generate proliferation of T cells. Generally, the naturally occurring full-length tumor antigen proteins do not display these properties and would thus not be useful for these immunotherapy purposes.

In certain embodiments, a tumor antigen-specific peptide (e.g., a VCX/Y peptide) is immunogenic or antigenic. As shown in the below examples, various tumor antigen-specific peptides (e.g., a VCX/Y peptide) of the present disclosure can promote the proliferation of T cells. It is anticipated that such peptides may be used to induce some degree of protective immunity.

A tumor antigen-specific peptide (e.g., a VCX/Y peptide) may be a recombinant peptide, synthetic peptide, purified peptide, immobilized peptide, detectably labeled peptide, encapsulated peptide, or a vector-expressed peptide (e.g., a peptide encoded by a nucleic acid in a vector comprising a heterologous promoter operably linked to the nucleic acid). In some embodiments, a synthetic tumor antigen-specific peptide (e.g., a VCX/Y peptide) may be administered to a subject, such as a human patient, to induce an immune response in the subject. Synthetic peptides may display certain advantages, such as a decreased risk of bacterial contamination, as compared to recombinantly expressed peptides. A tumor antigen-specific peptide (e.g., a VCX/Y peptide) may also be comprised in a pharmaceutical composition such as, e.g., a vaccine composition, which is formulated for administration to a mammalian or human subject.

A. Cell Penetrating Peptides

In some embodiments, an immunotherapy may utilize a tumor antigen-specific peptide (e.g., a VCX/Y peptide) of the present disclosure that is associated with a cell penetrator, such as a liposome or a cell penetrating peptide (CPP). Antigen presenting cells (such as dendritic cells) pulsed with peptides may be used to enhance antitumour immunity (Celluzzi et al., 1996; Young et al., 1996). Liposomes and CPPs are described in further detail below. In some embodiments, an immunotherapy may utilize a nucleic acid encoding a tumor antigen-specific peptide (e.g., a VCX/Y peptide) of the present disclosure, wherein the nucleic acid is delivered, e.g., in a viral vector or non-viral vector.

A tumor antigen-specific peptide (e.g., a VCX/Y peptide) may also be associated with or covalently bound to a cell penetrating peptide (CPP). Cell penetrating peptides that may be covalently bound to a tumor antigen-specific peptide (e.g., a VCX/Y peptide) include, e.g., HIV Tat, herpes virus VP22, the Drosophila Antennapedia homeobox gene product, signal sequences, fusion sequences, or protegrin I. Covalently binding a peptide to a CPP can prolong the presentation of a peptide by dendritic cells, thus enhancing antitumour immunity (Wang and Wang, 2002). In some embodiments, a tumor antigen-specific peptide (e.g., the VCX/Y peptide) of the present disclosure (e.g., comprised within a peptide or polyepitope string) may be covalently bound (e.g., via a peptide bond) to a CPP to generate a fusion protein. In other embodiments, a tumor antigen-specific peptide (e.g., a VCX/Y peptide) or nucleic acid encoding a tumor antigen-specific peptide may be encapsulated within or associated with a liposome, such as a mulitlamellar, vesicular, or multivesicular liposome, an exocytic vesicle or exosome.

As used herein, “association” means a physical association, a chemical association or both. For example, an association can involve a covalent bond, a hydrophobic interaction, encapsulation, surface adsorption, or the like.

As used herein, “cell penetrator” refers to a composition or compound which enhances the intracellular delivery of the peptide/polyepitope string to the antigen presenting cell. For example, the cell penetrator may be a lipid which, when associated with the peptide, enhances its capacity to cross the plasma membrane. Alternatively, the cell penetrator may be a peptide. Cell penetrating peptides (CPPs) are known in the art, and include, e.g., the Tat protein of HIV (Frankel and Pabo, 1988), the VP22 protein of HSV (Elliott and O'Hare, 1997) and fibroblast growth factor (Lin et al., 1995).

Cell-penetrating peptides (or “protein transduction domains”) have been identified from the third helix of the Drosophila Antennapedia homeobox gene (Antp), the HIV Tat, and the herpes virus VP22, all of which contain positively charged domains enriched for arginine and lysine residues (Schwarze et al., 2000; Schwarze et al., 1999). Also, hydrophobic peptides derived from signal sequences have been identified as cell-penetrating peptides. (Rojas et al., 1996; Rojas et al., 1998; Du et al., 1998). Coupling these peptides to marker proteins such as β-galactosidase has been shown to confer efficient internalization of the marker protein into cells, and chimeric, in-frame fusion proteins containing these peptides have been used to deliver proteins to a wide spectrum of cell types both in vitro and in vivo (Drin et al., 2002). Fusion of these cell penetrating peptides to a tumor antigen-specific peptide (e.g., a VCX/Y peptide) in accordance with the present disclosure may enhance cellular uptake of the polypeptides.

In some embodiments, cellular uptake is facilitated by the attachment of a lipid, such as stearate or myristilate, to the polypeptide. Lipidation has been shown to enhance the passage of peptides into cells. The attachment of a lipid moiety is another way that the present disclosure increases polypeptide uptake by the cell. Cellular uptake is further discussed below.

A tumor antigen-specific peptide (e.g., a VCX/Y peptide) of the present disclosure may be included in a liposomal vaccine composition. For example, the liposomal composition may be or comprise a proteoliposomal composition. Methods for producing proteoliposomal compositions that may be used with the present disclosure are described, e.g., in Neelapu et al. (2007) and Popescu et al. (2007). In some embodiments, proteoliposomal compositions may be used to treat a melanoma.

By enhancing the uptake of a tumor antigen-specific polypeptide, it may be possible to reduce the amount of protein or peptide required for treatment. This in turn can significantly reduce the cost of treatment and increase the supply of therapeutic agent. Lower dosages can also minimize the potential immunogencity of peptides and limit toxic side effects.

In some embodiments, a tumor antigen-specific peptide (e.g., a VCX/Y peptide) may be associated with a nanoparticle to form nanoparticle-polypeptide complex. In some embodiments, the nanoparticle is a liposomes or other lipid-based nanoparticle such as a lipid-based vesicle (e.g., a DOTAP:cholesterol vesicle). In other embodiments, the nanoparticle is an iron-oxide based superparamagnetic nanoparticles. Superparamagnetic nanoparticles ranging in diameter from about 10 to 100 nm are small enough to avoid sequestering by the spleen, but large enough to avoid clearance by the liver. Particles this size can penetrate very small capillaries and can be effectively distributed in body tissues. Superparamagnetic nanoparticles-polypeptide complexes can be used as MM contrast agents to identify and follow those cells that take up the tumor antigen-specific peptide (e.g., a VCX/Y peptide). In some embodiments, the nanoparticle is a semiconductor nanocrystal or a semiconductor quantum dot, both of which can be used in optical imaging. In further embodiments, the nanoparticle can be a nanoshell, which comprises a gold layer over a core of silica. One advantage of nanoshells is that polypeptides can be conjugated to the gold layer using standard chemistry. In other embodiments, the nanoparticle can be a fullerene or a nanotube (Gupta et al., 2005).

Peptides are rapidly removed from the circulation by the kidney and are sensitive to degradation by proteases in serum. By associating a tumor antigen-specific peptide (e.g., a VCX/Y peptide) with a nanoparticle, the nanoparticle-polypeptide complexes of the present disclosure may protect against degradation and/or reduce clearance by the kidney. This may increase the serum half-life of polypeptides, thereby reducing the polypeptide dose need for effective therapy. Further, this may decrease the costs of treatment, and minimizes immunological problems and toxic reactions of therapy.

B. Polyepitope Strings

In some embodiments, a tumor antigen-specific peptide (e.g., a VCX/Y peptide) is included or comprised in a polyepitope string. A polyepitope string is a peptide or polypeptide containing a plurality of antigenic epitopes from one or more antigens linked together. A polyepitope string may be used to induce an immune response in a subject, such as a human subject. Polyepitope strings have been previously used to target malaria and other pathogens (Baraldo et al., 2005; Moorthy et al., 2004; Baird et al., 2004). A polyepitope string may refer to a nucleic acid (e.g., a nucleic acid encoding a plurality of antigens including a VCX/Y peptide) or a peptide or polypeptide (e.g., containing a plurality of antigens including a VCX/Y peptide). A polyepitope string may be included in a cancer vaccine composition.

C. Biological Functional Equivalents

A tumor antigen-specific peptide (e.g., a VCX/Y peptide) of the present disclosure may be modified to contain amino acid substitutions, insertions and/or deletions that do not alter their respective interactions with an HLA class protein, such as HLA-A*0101, binding regions. Such a biologically functional equivalent of a tumor antigen-specific peptide (e.g., a VCX/Y peptide) could be a molecule having like or otherwise desirable characteristics, e.g., binding of HLA-A11, HLA-DB or HLA-DR As a nonlimiting example, certain amino acids may be substituted for other amino acids in a tumor antigen-specific peptide (e.g., a VCX/Y peptide) disclosed herein without appreciable loss of interactive capacity, as demonstrated by detectably unchanged peptide binding to HLA. In some embodiments, the tumor antigen-specific peptide has a substitution mutation at an anchor reside, such as a substitution mutation at one, two, or all of positions: 1 (P1), 2 (P2), and/or 9 (P9). It is thus contemplated that a tumor antigen-specific peptide (e.g., a VCX/Y peptide) disclosed herein (or a nucleic acid encoding such a peptide) which is modified in sequence and/or structure, but which is unchanged in biological utility or activity remains within the scope of the compositions and methods disclosed herein.

It is also well understood by the skilled artisan that, inherent in the definition of a biologically functional equivalent peptide, is the concept that there is a limit to the number of changes that may be made within a defined portion of the molecule while still maintaining an acceptable level of equivalent biological activity. Biologically functional equivalent peptides are thus defined herein as those peptides in which certain, not most or all, of the amino acids may be substituted. Of course, a plurality of distinct peptides with different substitutions may easily be made and used in accordance with the present disclosure.

The skilled artisan is also aware that where certain residues are shown to be particularly important to the biological or structural properties of a peptide, e.g., residues in specific epitopes, such residues may not generally be exchanged. This may be the case in the present disclosure, as a mutation in an tumor antigen-specific peptide (e.g., the VCX/Y peptide) disclosed herein could result in a loss of species-specificity and in turn, reduce the utility of the resulting peptide for use in methods of the present disclosure. Thus, peptides which are antigenic (e.g., bind HLA-A11, HLA-DB or HLA-DR specifically) and comprise conservative amino acid substitutions are understood to be included in the present disclosure. Conservative substitutions are least likely to drastically alter the activity of a protein. A “conservative amino acid substitution” refers to replacement of amino acid with a chemically similar amino acid, i.e., replacing nonpolar amino acids with other nonpolar amino acids; substitution of polar amino acids with other polar amino acids, acidic residues with other acidic amino acids, etc.

Amino acid substitutions, such as those which might be employed in modifying a tumor antigen-specific peptide (e.g., a VCX/Y peptide) disclosed herein are generally based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents. In some embodiments, the mutation may enhance TCR-pMHC interaction and/or peptide-MHC binding.

The present disclosure also contemplates isoforms of the tumor antigen-specific peptides (e.g., a VCX/Y peptide) disclosed herein. An isoform contains the same number and kinds of amino acids as a peptide of the present disclosure, but the isoform has a different molecular structure. The isoforms contemplated by the present disclosure are those having the same properties as a peptide of the present disclosure as described herein.

Nonstandard amino acids may be incorporated into proteins by chemical modification of existing amino acids or by de novo synthesis of a peptide disclosed herein. A nonstandard amino acid refers to an amino acid that differs in chemical structure from the twenty standard amino acids encoded by the genetic code.

In select embodiments, the present disclosure contemplates a chemical derivative of a tumor antigen-specific peptide (e.g., a VCX/Y peptide) disclosed herein. “Chemical derivative” refers to a peptide having one or more residues chemically derivatized by reaction of a functional side group, and retaining biological activity and utility. Such derivatized peptides include, for example, those in which free amino groups have been derivatized to form specific salts or derivatized by alkylation and/or acylation, p-toluene sulfonyl groups, carbobenzoxy groups, t-butylocycarbonyl groups, chloroacetyl groups, formyl or acetyl groups among others. Free carboxyl groups may be derivatized to form organic or inorganic salts, methyl and ethyl esters or other types of esters or hydrazides and preferably amides (primary or secondary). Chemical derivatives may include those peptides which comprise one or more naturally occurring amino acids derivatives of the twenty standard amino acids. For example, 4-hydroxyproline may be substituted for serine; and ornithine may be substituted for lysine.

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. The amino acids described herein are preferred to be in the “L” isomeric form. However, residues in the “D” isomeric form can be substituted for any L-amino acid residue, as long as the desired functional properties set forth herein are retained by the protein.

Preferred tumor antigen-specific peptides (e.g., a VCX/Y peptide) or analogs thereof preferably specifically or preferentially bind a HLA-A11, HLA-DB or HLA-DR Determining whether or to what degree a particular tumor antigen-specific peptide or labeled peptide, or an analog thereof, can bind an HLA-A11, HLA-DB or HLA-DR and can be assessed using an in vitro assay such as, for example, an enzyme-linked immunosorbent assay (ELISA), immunoblotting, immunoprecipitation, radioimmunoassay (MA), immunostaining, latex agglutination, indirect hemagglutination assay (IHA), complement fixation, indirect immnunofluorescent assay (FA), nephelometry, flow cytometry assay, chemiluminescence assay, lateral flow immunoassay, u-capture assay, mass spectrometry assay, particle-based assay, inhibition assay and/or an avidity assay.

D. Nucleic Acids Encoding a Tumor Antigen-Specific Peptide

In an aspect, the present disclosure provides a nucleic acid encoding an isolated antigen-specific peptide comprising a sequence that has at least about 90%, 91%, 92%, 93%, 94%, 95%, 96%, 97%, 98%, 99%, or 100% sequence identity SEQ ID NOs: 1-15, or the peptide may have 1, 2, 3, or 4 point mutations (e.g., substitution mutations) as compared to SEQ ID NO: 1-15. As stated above, such a tumor antigen-specific peptide may be, e.g., from 8 to 35 amino acids in length, or any range derivable therein. In some embodiments, the tumor antigen-specific peptide corresponds to a portion of the tumor antigen protein such as VCX1, VCX2, VCX3A, VCX3B, or VCY (e.g., VCX3A; GenBank Accession No: AAI26903.1). The term “nucleic acid” is intended to include DNA and RNA and can be either double stranded or single stranded.

Some embodiments of the present disclosure provide recombinantly-produced tumor antigen-specific peptides (e.g., a VCX/Y peptide) which can specifically bind a HLA-A11, HLA-DB or HLA-DR. Accordingly, a nucleic acid encoding a tumor antigen-specific peptide may be operably linked to an expression vector and the peptide produced in the appropriate expression system using methods well known in the molecular biological arts. A nucleic acid encoding a tumor antigen-specific peptide disclosed herein may be incorporated into any expression vector which ensures good expression of the peptide. Possible expression vectors include but are not limited to cosmids, plasmids, or modified viruses (e.g. replication defective retroviruses, adenoviruses and adeno-associated viruses), so long as the vector is suitable for transformation of a host cell.

A recombinant expression vector being “suitable for transformation of a host cell” means that the expression vector contains a nucleic acid molecule of the present disclosure and regulatory sequences selected on the basis of the host cells to be used for expression, which is operatively linked to the nucleic acid molecule. The terms, “operatively linked” or “operably linked” are used interchangeably and are intended to mean that the nucleic acid is linked to regulatory sequences in a manner which allows expression of the nucleic acid.

Accordingly, the present disclosure provides a recombinant expression vector comprising nucleic acid encoding a tumor antigen-specific peptide, and the necessary regulatory sequences for the transcription and translation of the inserted protein-sequence. Suitable regulatory sequences may be derived from a variety of sources, including bacterial, fungal, or viral genes (e.g., see the regulatory sequences described in Goeddel (1990).

Selection of appropriate regulatory sequences is generally dependent on the host cell chosen, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector. It will also be appreciated that the necessary regulatory sequences may be supplied by the native protein and/or its flanking regions.

A recombinant expression vector may also contain a selectable marker gene which facilitates the selection of host cells transformed or transfected with a recombinant tumor antigen-specific peptides (e.g., a VCX/Y peptide) disclosed herein. Examples of selectable marker genes are genes encoding a protein such as G418 and hygromycin which confer resistance to certain drugs, β-galactosidase, chloramphenicol acetyltransferase, or firefly luciferase. Transcription of the selectable marker gene is monitored by changes in the concentration of the selectable marker protein such as β-galactosidase, chloramphenicol acetyltransferase, or firefly luciferase. If the selectable marker gene encodes a protein conferring antibiotic resistance such as neomycin resistance transformant cells can be selected with G418. Cells that have incorporated the selectable marker gene will survive, while the other cells die. This makes it possible to visualize and assay for expression of a recombinant expression vector, and in particular, to determine the effect of a mutation on expression and phenotype. It will be appreciated that selectable markers can be introduced on a separate vector from the nucleic acid of interest.

Recombinant expression vectors can be introduced into host cells to produce a transformant host cell. The term “transformant host cell” is intended to include prokaryotic and eukaryotic cells which have been transformed or transfected with a recombinant expression vector of the present disclosure. The terms “transformed with”, “transfected with”, “transformation” and “transfection” are intended to encompass introduction of nucleic acid (e.g. a vector) into a cell by one of many possible techniques known in the art. Suitable host cells include a wide variety of prokaryotic and eukaryotic host cells. For example, the proteins of the present disclosure may be expressed in bacterial cells such as E. coli, insect cells (using baculovirus), yeast cells or mammalian cells.

A nucleic acid molecule of the present disclosure may also be chemically synthesized using standard techniques. Various methods of chemically synthesizing polydeoxy-nucleotides are known, including solid-phase synthesis which, like peptide synthesis, has been fully automated in commercially available DNA synthesizers (See e.g., U.S. Pat. Nos. 4,598,049; 4,458,066; 4,401,796; and 4,373,071).

### III. ANTIGEN-SPECIFIC CELL THERAPY

Embodiments of the present disclosure concern obtaining and administering antigen-specific cells (e.g., autologous or allogeneic T cells (e.g., regulatory T cells, CD4⁺ T cells, CD8⁺ T cells, or gamma-delta T cells), NK cells, invariant NK cells, NKT cells, mesenchymal stem cell (MSC)s, or induced pluripotent stem (iPS) cells) to a subject as an immunotherapy to target cancer cells. In particular, the cells are antigen-specific T cells (e.g., VCX/Y-specific T cells). Several basic approaches for the derivation, activation and expansion of functional anti-tumor effector cells have been described in the last two decades. These include: autologous cells, such as tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. These approaches have given rise to numerous protocols for T cell preparation and immunization which can be used in the methods described herein.

A. T Cell Preparation

In some embodiments, the T cells are derived from the blood, bone marrow, lymph, umbilical cord, or lymphoid organs. In some aspects, the cells are human cells. The cells typically are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells (e.g., CD4⁺ and/or CD8⁺ T cells) are naive T (T_{N}) cells, effector T cells (T_{EFF}), memory T cells and sub-types thereof, such as stem cell memory T (TSC_{M}), central memory T (TC_{M}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, one or more of the T cell populations is enriched for or depleted of cells that are positive for a specific marker, such as surface markers, or that are negative for a specific marker. In some cases, such markers are those that are absent or expressed at relatively low levels on certain populations of T cells (e.g., non-memory cells) but are present or expressed at relatively higher levels on certain other populations of T cells (e.g., memory cells).

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ T cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al., 2012; Wang et al., 2012.

In some embodiments, the T cells are autologous T cells. In this method, tumor samples are obtained from patients and a single cell suspension is obtained. The single cell suspension can be obtained in any suitable manner, e.g., mechanically (disaggregating the tumor using, e.g., a gentleMACS™ Dissociator, Miltenyi Biotec, Auburn, Calif.) or enzymatically (e.g., collagenase or DNase). Single-cell suspensions of tumor enzymatic digests are cultured in interleukin-2 (IL-2). The cells are cultured until confluence (e.g., about 2×10⁶ lymphocytes), e.g., from about 5 to about 21 days, preferably from about 10 to about 14 days.

The cultured T cells can be pooled and rapidly expanded. Rapid expansion provides an increase in the number of antigen-specific T-cells of at least about 50-fold (e.g., 50-, 60-, 70-, 80-, 90-, or 100-fold, or greater) over a period of about 10 to about 14 days. More preferably, rapid expansion provides an increase of at least about 200-fold (e.g., 200-, 300-, 400-, 500-, 600-, 700-, 800-, 900-, or greater) over a period of about 10 to about 14 days.

Expansion can be accomplished by any of a number of methods as are known in the art. For example, T cells can be rapidly expanded using non-specific T cell receptor stimulation in the presence of feeder lymphocytes and either interleukin-2 (IL-2) or interleukin-15 (IL-15), with IL-2 being preferred. The non-specific T cell receptor stimulus can include around 30 ng/ml of OKT3, a mouse monoclonal anti-CD3 antibody (available from Ortho-McNeil®, Raritan, N.J.). Alternatively, T cells can be rapidly expanded by stimulation of PBMCs in vitro with one or more antigens (including antigenic portions thereof, such as epitope(s), or a cell) of the cancer, which can be optionally expressed from a vector, such as an human leukocyte antigen A2 (HLA-A2) binding peptide, in the presence of a T cell growth factor, such as 300 IU/ml IL-2 or IL-15, with IL-2 being preferred. The in vitro-induced T cells are rapidly expanded by re-stimulation with the same antigen(s) of the cancer pulsed onto HLA-A2-expressing antigen-presenting cells. Alternatively, the T cells can be re-stimulated with irradiated, autologous lymphocytes or with irradiated HLA-A2+ allogeneic lymphocytes and IL-2, for example.

The autologous T cells can be modified to express a T cell growth factor that promotes the growth and activation of the autologous T cells. Suitable T cell growth factors include, for example, IL-2, IL-7, IL-15, and IL-12. Suitable methods of modification are known in the art. See, for instance, Sambrook et al., 2001; and Ausubel et al., 1994. In particular aspects, modified autologous T cells express the T cell growth factor at high levels. T cell growth factor coding sequences, such as that of IL-12, are readily available in the art, as are promoters, the operable linkage of which to a T cell growth factor coding sequence promote high-level expression.

The present T cells, such as the starting population of T cells, may be engineered T cells. In certain embodiments, the engineered T cells comprise T cells expressing a chimeric antigen receptor (CAR T cells). In certain embodiments, the engineered T cells comprise T cells expressing a recombinant T cell receptor capable of binding tumor-specific epitopes or neoepitopes. In some embodiments, the engineered T cells are constructed using any of the many well-established gene transfer methods known to those skilled in the art. In certain embodiments, the engineered cells are constructed using viral vector-based gene transfer methods to introduce nucleic acids encoding a chimeric antigen receptor specific for a desired target tumor antigen or encoding a recombinant TCR specific for a desired tumor-specific epitope or neoepitope. In certain embodiments, the engineered cells are constructed using non-viral vector-based gene transfer methods to introduce nucleic acids encoding a chimeric antigen receptor specific for a desired target tumor antigen or encoding a recombinant TCR specific for a desired tumor-specific epitope or neoepitope. In certain embodiments, the viral vector-based gene transfer method comprises a lentiviral vector. In certain embodiments, the viral vector-based gene transfer method comprises a retroviral vector. In certain embodiments, the viral vector-based gene transfer method comprises an adenoviral or an adeno-associated viral vector. In certain embodiments, the non-viral vector-based gene transfer method comprises a gene-editing method selected from the group consisting of a zinc-finger nuclease (ZFN), a transcription activator-like effector nuclease (TALENs), and a clustered regularly interspaced short palindromic repeats (CRISPR)/CRISPR-associated protein 9 (Cas9) nuclease. In certain embodiments, the non-viral vector-based gene editing method comprises a transfection or transformation method selected from the group consisting of lipofection, nucleofection, biolistics, virosomes, liposomes, polycation or lipid:nucleic acid conjugates, naked DNA, artificial virions, and agent-enhanced uptake of DNA.

In certain embodiments, the CAR T cell expresses a CAR construct comprising an extracellular antigen-binding domain, an optional spacer sequence, a transmembrane domain, one or more intracellular signaling domains, and one or more optional regulatory sequences for activating or inactivating the CAR T cell.

In certain embodiments, the extracellular antigen-binding domain comprises a moiety capable of specifically binding a desired target. In certain embodiments, the moiety capable of specifically binding a desired target comprises a monoclonal antibody or antigen-binding fragment thereof. In certain embodiments, the antigen-binding fragment thereof comprises a single-chain variable fragment (scFv) of a monoclonal antibody capable of specifically binding a desired target. In certain embodiments, the desired target is a tumor-specific antigen.

In certain embodiments, the transmembrane domain comprises any synthetic or natural amino acid sequence capable of forming a structure able to span a cell membrane. In certain embodiments, the structure able to span a cell membrane comprises an alpha helix. In certain embodiments, the transmembrane region is derived from a naturally occurring transmembrane protein selected from the group consisting of CD3, CD3c, CD4, CD5, CD8, CD9, CD16, CD22, CD28, CD33, CD37, CD45, CD64, CD80, CD86, CD134, 4-1BB/CD137, CD154, inducible T cell costimulator (ICOS)/CD278, glucocorticoid-induced TNFR-related protein (GITR)/CD357, NKG2D, TCRα and TCRβ. In certain embodiments, the transmembrane region derived from a naturally occurring transmembrane protein comprises one or more amino acid substitutions in sequences known to be involved in interactions with other signaling proteins.

In certain embodiments, the one or more intracellular signaling domains comprise one or more intracellular tyrosine-based activation motifs (“ITAMs”). In certain embodiments, the one or more ITAMs are present on a CD3-zeta (CD3ζ) molecule. In certain embodiments, the one or more intracellular signaling domains further comprise a costimulatory signaling domain selected from the group consisting of CD28, 4-1BB/CD137, ICOS, OX40, CD2, CD40L, CD27, Light-R, GITR, or combinations thereof.

In certain embodiments, the T cells comprise a recombinant T cell receptor capable of binding tumor-specific epitopes or neoepitopes, e.g., of a VCX/Y polypeptide. In certain embodiments, the recombinant T cell receptor comprises a naturally occurring TCR cloned from a T cell isolated from a subject. In certain embodiments, the recombinant TCR comprises a heterodimer comprising a TCR alpha (TCRα) polypeptide and a TCR beta (TCRβ) polypeptide (i.e., a TCRαβ). In certain embodiments, the recombinant TCR comprises a heterodimer comprising a TCR gamma (TCRγ) polypeptide and a TCR delta (TCRδ) polypeptide (i.e., a TCRγδ).

In certain embodiments, the recombinant TCRαβ comprises a cloned TCRαβ isolated from a subject and specific for a peptide antigen derived from a desired target. In certain embodiments, the subject is a mammal. In certain embodiments, the mammal is a human. In certain embodiments, the desired target is a VCX/Y polypeptide. In certain embodiments, the recombinant TCRγδ comprises a cloned TCRγδ isolated from a subject and specific for a peptide antigen derived from a desired target. In certain embodiments, the subject is a mammal. In certain embodiments, the mammal is a human.

As used herein, the terms “chimeric antigen receptor”, “CAR”, “chimeric T cell receptor”, “artificial T cell receptor” or “chimeric immunoreceptor” refer to an engineered chimeric receptor construct grafting a desired non-MHC-restricted antigen-binding specificity onto an immune effector cell, e.g., an effector T cell. CARs may comprise, for example, an extracellular antigen-binding domain (e.g., an antibody or an antibody fragment such as, for example, a single-chain variable fragment (scFv) having the desired antigen specificity), a spacer sequence, a transmembrane domain, and one or more intracellular signaling domains. Exemplary intracellular signaling domains may comprise one or more intracellular tyrosine-based activation motifs (“ITAMs”), such as CD3-zeta (CD3ζ), and/or one or more costimulatory signaling domains, such as, for example, CD28, 4-1BB/CD137, ICOS, OX40, or combinations thereof.

B. Antigen-Presenting Cells

Antigen-presenting cells, which include macrophages, B lymphocytes, and dendritic cells, are distinguished by their expression of a particular MHC molecule. APCs internalize antigen and re-express a part of that antigen, together with the MHC molecule on their outer cell membrane. The major histocompatibility complex (MHC) is a large genetic complex with multiple loci. The MHC loci encode two major classes of MHC membrane molecules, referred to as class I and class II MHCs. T helper lymphocytes generally recognize antigen associated with MHC class II molecules, and T cytotoxic lymphocytes recognize antigen associated with MHC class I molecules. In humans the MHC is referred to as the HLA complex and in mice the H-2 complex. In some aspects, a VCX/Y peptides of the embodiments are expressed in antigen presenting cells. Such cells provide engineered APCs that can be used to specifically propagate immune effector cells specific for the VCX/Y antigen of interest.

In some cases, aAPCs are useful in preparing therapeutic compositions and cell therapy products of the embodiments. For general guidance regarding the preparation and use of antigen-presenting systems, see, e.g., U.S. Pat. Nos. 6,225,042, 6,355,479, 6,362,001 and 6,790,662; U.S. Patent Application Publication Nos. 2009/0017000 and 2009/0004142; and International Publication No. WO2007/103009.

aAPC systems may comprise at least one exogenous assisting molecule. Any suitable number and combination of assisting molecules may be employed. The assisting molecule may be selected from assisting molecules such as co-stimulatory molecules and adhesion molecules. Exemplary co-stimulatory molecules include CD86, CD64 (FcγRI), 41BB ligand, and IL-21. Adhesion molecules may include carbohydrate-binding glycoproteins such as selectins, transmembrane binding glycoproteins such as integrins, calcium-dependent proteins such as cadherins, and single-pass transmembrane immunoglobulin (Ig) superfamily proteins, such as intercellular adhesion molecules (ICAMs), which promote, for example, cell-to-cell or cell-to-matrix contact. Exemplary adhesion molecules include LFA-3 and ICAMs, such as ICAM-1. Techniques, methods, and reagents useful for selection, cloning, preparation, and expression of exemplary assisting molecules, including co-stimulatory molecules and adhesion molecules, are exemplified in, e.g., U.S. Pat. Nos. 6,225,042, 6,355,479, and 6,362,001.

### IV. METHODS OF TREATMENT

Provided herein are methods for treating or delaying progression of cancer in an individual comprising administering to the individual an effective amount an antigen-specific cell therapy, such as a VCX/Y-specific T cell therapy. Adoptive T cell therapies with genetically engineered TCR-transduced T cells (conjugate TCR to other bioreactive proteins (e.g., anti-CD3) are also provided herein. In further embodiments, methods are provided for the treatment of cancer comprising immunizing a subject with a purified tumor antigen or an immunodominant tumor antigen-specific peptide.

The VCX/Y peptide provided herein can be utilized to develop cancer vaccines or immunogens (e.g., a peptide or modified peptide mix with adjuvant, coding polynucleotide and corresponding expression products such as inactive virus or other microorganisms vaccine). These peptide specific vaccines or immunogens can be used for immunizing cancer patients directly to induce anti-tumor immuno-response in vivo, or for expanding antigen specific T cells in vitro with peptide or coded polynucleotide loaded APC stimulation. These large number of T cells can be adoptively transferred to patients to induce tumor regression.

Examples of cancers contemplated for treatment include lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon cancer, melanoma, and bladder cancer.

In some embodiments, T cells are autologous. However the cells can be allogeneic. In some embodiments, the T cells are isolated from the patient themself, so that the cells are autologous. If the T cells are allogeneic, the T cells can be pooled from several donors. The cells are administered to the subject of interest in an amount sufficient to control, reduce, or eliminate symptoms and signs of the disease being treated.

In some embodiments, the subject can be administered nonmyeloablative lymphodepleting chemotherapy prior to the T cell therapy. The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine, particularly if the cancer is melanoma, which can be metastatic. An exemplary route of administering cyclophosphamide and fludarabine is intravenously. Likewise, any suitable dose of cyclophosphamide and fludarabine can be administered. In particular aspects, around 60 mg/kg of cyclophosphamide is administered for two days after which around 25 mg/m² fludarabine is administered for five days.

In certain embodiments, a T-cell growth factor that promotes the growth and activation of the autologous T cells is administered to the subject either concomitantly with the autologous T cells or subsequently to the autologous T cells. The T-cell growth factor can be any suitable growth factor that promotes the growth and activation of the autologous T-cells. Examples of suitable T-cell growth factors include IL-2, IL-7, IL-15, and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2. IL-12 is a preferred T-cell growth factor.

The T cell may be administered intravenously, intramuscularly, subcutaneously, topically, orally, transdermally, intraperitoneally, intraorbitally, by implantation, by inhalation, intrathecally, intraventricularly, or intranasally. The appropriate dosage of the T cell therapy may be determined based on the type of disease to be treated, severity and course of the disease, the clinical condition of the individual, the individual's clinical history and response to the treatment, and the discretion of the attending physician.

Intratumoral injection, or injection into the tumor vasculature is specifically contemplated for discrete, solid, accessible tumors. Local, regional or systemic administration also may be appropriate. For tumors of >4 cm, the volume to be administered will be about 4-10 ml (in particular 10 ml), while for tumors of <4 cm, a volume of about 1-3 ml will be used (in particular 3 ml). Multiple injections delivered as single dose comprise about 0.1 to about 0.5 ml volumes.

A. Pharmaceutical Compositions

Also provided herein are pharmaceutical compositions and formulations comprising antigen-specific immune cells (e.g., T cells) or receptors (e.g., TCR) and a pharmaceutically acceptable carrier. A vaccine composition for pharmaceutical use in a subject may comprise a tumor antigen peptide (e.g., VCX/Y) composition disclosed herein and a pharmaceutically acceptable carrier.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn— protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

B. Combination Therapies

In certain embodiments, the compositions and methods of the present embodiments involve an antigen-specific immune cell population or TCR in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

An immune cell therapy may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an antigen-specific immune cell therapy, peptide, or TCR is “A” and an anti-cancer therapy is

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Application No. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUIDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### V. ARTICLES OF MANUFACTURE OR KITS

An article of manufacture or a kit is provided comprising antigen-specific immune cells, TCRs, or antigen peptides (e.g., VCX/Y peptide) is also provided herein. The article of manufacture or kit can further comprise a package insert comprising instructions for using the antigen-specific immune cells to treat or delay progression of cancer in an individual or to enhance immune function of an individual having cancer. Any of the antigen-specific immune cells described herein may be included in the article of manufacture or kits. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Identification and Characterization of Tumor Antigen-Specific Peptides

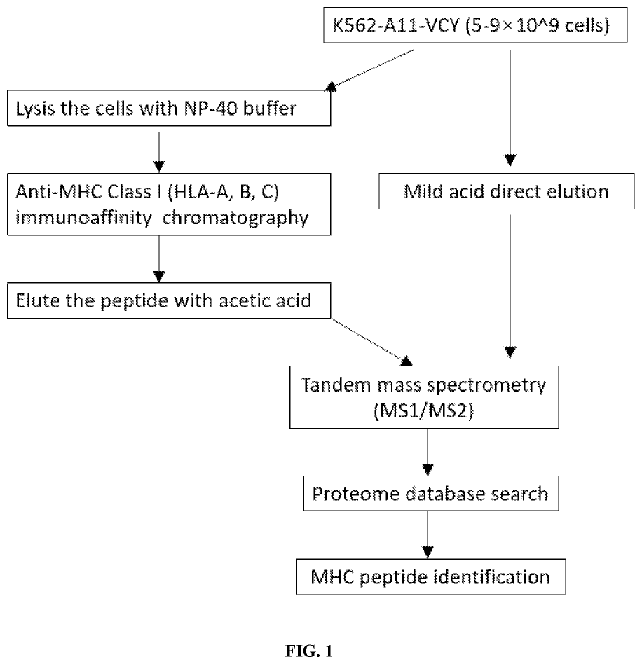

VCX/Y HLA-A11 restricted peptides were identified by eluting MHC binding peptides from tumor cells followed by tandem mass spectrum analysis. A schematic of the experimental approach for these studies is provided as FIG. 1.

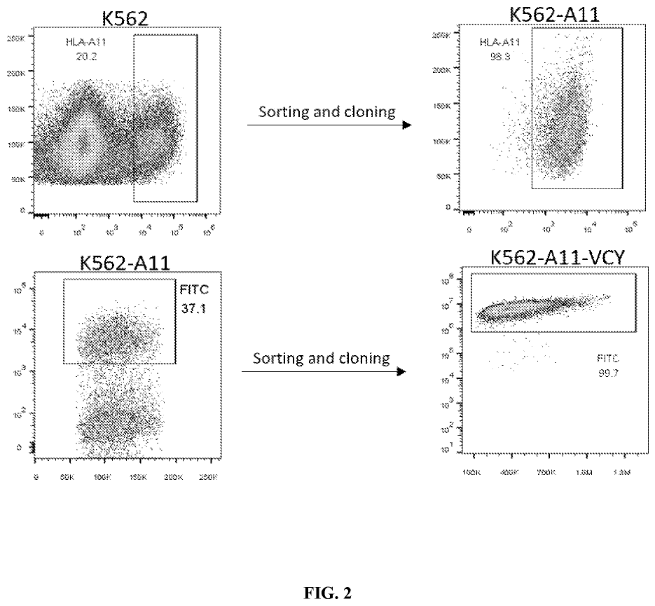

First, the HLA-A11 gene was introduced into the K562 parental cell line using a lentiviral vector. After infection with the vector, the HLA-A11 positive population was sorted using fluorescence assisted cell sorting (FACS; see, FIG. 2) and the sorted cells were cultured to allow for clonal selection. Various clonal populations were screened and HLA-A11 overexpressing clone (K562-A11) was selected and further expanded. Subsequently, a VCY fusion to eGFP was introduced into K562-A11 using a lentiviral vector. After infection, the GFP positive population was sorted using FACS and a GFP positive clone (K562-A11-VCY) was selected and further expanded. The K562-A11-VCY cell line that was generated exhibits forced expression of both HLA-A11 and VCY. Because this cell line only expresses one HLA allele, all MHC peptide processed and presented by this cell line are HLA-A11 restricted. By utilizing this approach, the possibility of identifying more HLA-A11 restricted peptides, especially for those with low abundance, is maximized.

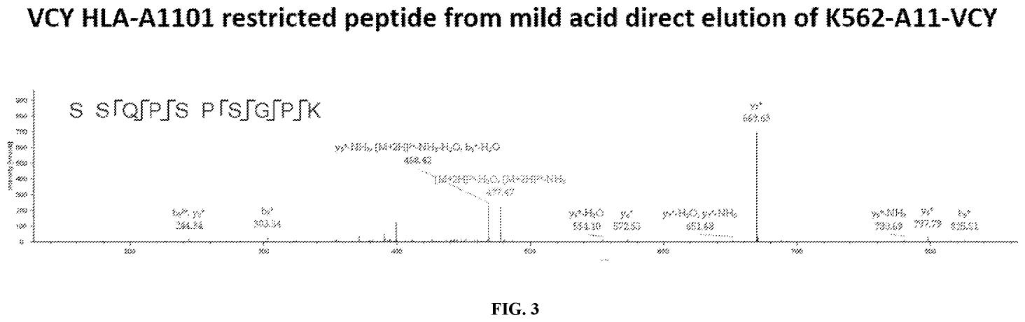

Two elution methods were used for peptide elution and identification. The first method is mild acid direct elution. MHC peptides on the cell surface were eluted directly using mild acid (citrate-phosphate buffer, pH 3.3) from 4-5×10⁸ K562-A11-VCY cells. The peptide solutions were desalted with C18 column, concentrated with spin-vacuum and analyzed using tandem mass spectrometry (MS1/MS2). The results of these analyses are shown in FIG. 3. One of the peptides identified (SSQPSPSGPK; SEQ ID NO: 2) matched the VCY sequence. Details on the peptide are provided in Table 1. From the NetMHC HLA binding prediction tool, the binding affinity of this peptide to HLA-A1101 is predicted to be 33.85 nM, making it a predicted strong HLA-A11 binding peptide.

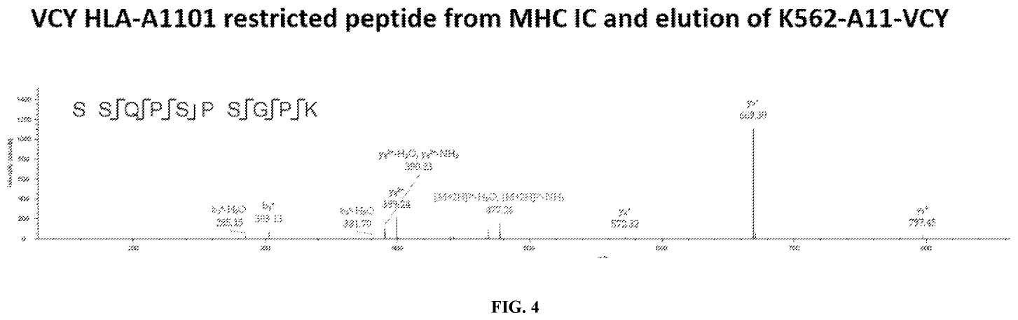

The second method used for peptide identification was an MHC immunoaffinity chromatography (MHC IC) method. Approximately 1×10⁹ K562-A11-VCY cells were lysed with NP-40 lysis buffer and the lysate was cleared by centrifuge sedimentation and filtering and then incubated with Sepharose Fast Flow beads coupled to an anti-MHC class I antibody (anti-HLA-A, B, C, W6/32 clone). After allowing for binding, the unbound protein was washed out with PBS and the MHC binding peptides were eluted with 5% acetic acid. The peptide solution was cleared with ultrafiltering, concentrated by spin-vacuum and analyzed using tandem mass spectrometry (MS1/MS2) (see FIG. 4). One of the peptides identified (SSQPSPSGPK; SEQ ID NO: 2) is part of the VCY sequence. Details of the peptide are provided in Table 1; this peptide was identical to the peptide identified in the elution method. By MHW IC elution and MS analysis, another peptide identified (ASGPPAKAK; SEQ ID NO: 1) also corresponds to the sequence of VCY. The ion score for this peptide is 41 as indicated in Table 1. From the NetMHC HLA binding prediction algorithm, the binding affinity of this peptide to HLA-A1101 is 250.51 nM, indicating it is a predicted weak HLA-A11 binding peptide.

TABLE 1
Summary of VCX/Y HLA-A11 restricted peptides eluted
from K562-A11-VCY.

| | | | NetMHC | Elution | VCX/Y |
|---|---|---|---|---|---|
| Position | Sequence | Ion Score | binding | methods | member |
| VCY-23-32 | SSQPSPSGPK; | 41 | 33.85 nM | Mild Acid | VCY |
| | SEQ ID NO: 2 | | | | |
| VCY-23-32 | SSQPSPSGPK; | 34 | 33.85 nM | MHC IC | VCY |
| | SEQ ID NO: 2 | | | | |
| VCY-7-15 | ASGPPAKAK; | 41 | 250.51 nM | MHC IC | VCY, |
| | SEQ ID NO: 1 | | | | VCX3B |

Table 2 shows long peptide sequences (amino acids 1-32) from VCY and VCX3B, that for VCY encompasses both peptides (ASGPPAKAK; SEQ ID NO: 1 and SSQPSPSGPK; SEQ ID NO: 2) identified by elution and M/S. The sequence of VCX3B also comprises one of the peptides identified by elution and M/S (ASGPPAKAK; SEQ ID NO: 1), but the sequence corresponding to the second peptide differs from VCY by one amino acid (bolded and underlined in Table 2). The peptide SSQPSPSDPK (SEQ ID NO: 3; VCX-23-32) is very similar to VCY-23-32 (SSQPSPSGPK; SEQ ID NO: 2).

TABLE 2
Long peptide encompassing VCY-7-15 and
VCY-23-32 and corresponding VCX3B sequences.

| | | VCX/Y |
|---|---|---|
| Position | Sequence | member |
| VCY-1-32 | MSPKPRASGPPAKAKETGKRKSSSQPSPSGPK; | VCY |
| | SEQ ID NO: 6 | |
| VCX-1-32 | MSPKPRASGPPAKAKETGKRKSSSQPSPSDPK; | VCX3B |
| | SEQ ID NO: 7 | |

Table 3 shows a comparison of VCX-23-32 and VCY-23-32. From the NetMHC binding prediction, the binding affinity of VCX-23-32 peptide to HLA-A11 is 42.02 nM, still predicted to be a strong HLA-A11 binding peptide. The VCY-23-32 peptide is only shared by VCY. The VCX-23-32 peptide is common to VCX1, VCX2, VCX3A and VCX3B. Thus, the VCX-23-32 peptide may be valuable HLA-A11 restricted peptide targeting four VCX genes.

TABLE 3
Comparison of VCX-23-32 peptide to
VCY-23-32 peptide.

| | | NetMHC | VCX/Y |
|---|---|---|---|
| Position | Sequence | binding | member |
| VCY-23- | SSQPSPSGPK; SEQ ID NO: 2 | 33.85 nM | VCY |
| 32 | | | |
| VCX-23- | SSQPSPSDPK; SEQ ID NO: 3 | 42.02 nM | VCX1, |
| 32 | | | VCX2, |
| | | | VCX3A, |
| | | | VCX3B |

Table 4 shows the predicted MHC binding of Class II restricted peptides found in long peptide encompassing both of the HLA-A11 peptides. Class II restricted epitopes can bind strongly to more prevalent HLA-DR, -DQ allotypes, collectively covering >50% of the human population. The long peptides contain both Class I epitopes and Class II epitopes, which suggests the induction of CD4+ and CD8+ T cell expansion at the same time. Thus, the long peptide identified for VCY and VCX in Table 2 could be an ideal candidate to induce T cell response. Table 5 provides a further predicted HLA-binding peptide from VCX/Y (shared between VCX1, 2, 3A, 3B and VCY).

TABLE 4
Predicted Class II binding epitopes in long peptide
encompassing both HLA-A11 peptide.

| | | | | Affinity | |
|---|---|---|---|---|---|
| Allele | Pos | Peptide | Core | (nM) | Frequency |
| HLA- | 1 | MSPKPRASGPPAKAK | RASGPPAKA | 81.2 | 40% |
| DQA10501- | | (SEQ ID NO: 12) | (SEQ ID NO: 8) | | |
| DQB10301 | | | | | |
| DRB1_1301 | 8 | SGPPAKAKETGKRKS | AKAKETGKR | 193.6 | 12% |
| | | (SEQ ID NO: 13) | (SEQ ID NO: 9) | | |
| DRB1_1301 | 11 | PAKAKETGKRKSSSQ | AKAKETGKR | 148.8 | 12% |
| | | (SEQ ID NO: 14) | (SEQ ID NO: 9) | | |
| DRB5_0101 | 11 | PAKAKETGKRKSSSQ | AKAKETGKR | 58.2 | 8% |
| | | (SEQ ID NO: 14) | (SEQ ID NO: 9) | | |
| HLA- | 48 | GRRGKKGAATKMAAV | KGAATKMAA | 86.5 | 40% |
| DQA10501- | | (SEQ ID NO: 15) | (SEQ ID NO: | | |
| DQB10301 | | | 10) | | |
| HLA- | 48 | GRRGKKGAATKMAAV | GAATKMAAV | 41.0 | 10% |
| DQA10102- | | (SEQ ID NO: 15) | (SEQ ID NO: | | |
| DQB10602 | | | 11) | | |

TABLE 5
Additional predicted Class II binding epitopes from VCX/Y

| | | | | Affinity | |
|---|---|---|---|---|---|
| Allele | Pos | Peptide | Core | (nM) | Frequency |
| HLA- | 48 | GRRGKKGAATKMAAV | KGAATKMAA | 86.5 | 40% |
| DQA10501- | | (SEQ ID NO: 15) | (SEQ ID NO: | | |
| DQB10301 | | | 10) | | |
| HLA- | 48 | GRRGKKGAATKMAAV | GAATKMAAV | 41.0 | 10% |
| DQA10102- | | (SEQ ID NO: 15) | (SEQ ID NO: | | |
| DQB10602 | | | 11) | | |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Austin-Ward and Villaseca, Revista Medica de Chile, 126(7):838-845, 1998.
Ausubel et al., Current Protocols in Molecular Biology, Greene Publishing Associates and
John Biology Publications, p. 433, 1997.
Baird et al., Scand. J Immunol., 60(4):363-71, 2004.
Baraldo et al., Infect. Immun., 73(9):5835-41, 2005.
Bijker et al., J. Immunol., 179:5033-5040, 2007.
Blanchard and Shastri, Curr. Opin. Immunol., 20:82-88, 2008.
Bukowski et al., Clinical Cancer Res., 4(10):2337-2347, 1998.
Burrows et al., Trends Immunol., 27:11-16, 2006.
Camacho et al. (2004) J Clin Oncology 22(145): Abstract No. 2505, 2004.
Celluzzi et al., J. Exp. Med., 183 283-287, 1996.
Chothia et al., EMBO J. 7:3745, 1988.
Christodoulides et al., Microbiology, 144(Pt 11):3027-3037, 1998.
Cohen et al. J Immunol. 175:5799-5808, 2005.
Collins et al., Nature, 371:626-629, 1994.
Davidson et al., J. Immunother., 21(5):389-398, 1998.
Davila et al. PLoS ONE 8(4): e61338, 2013.
Drin et al., AAPS Pharm. Sci., 4(4):E26, 2002.
Du et al., J. Pept. Res., 51:235-243, 1998.
Dudley et al., J. Immunol., 26(4):332-342, 2003.
Elliott and O'Hare, Cell, 88:23-233, 1997.
European Patent Application No. EP2537416
Fedorov et al., Sci. Transl. Medicine, 5(215) 2013.
Janeway et al, Immunobiology: The Immune System in Health and Disease, 3^{rd} Ed., Current
Frankel and Pabo, Cell, 55:189-1193, 1988.
Goeddel, Methods Enzymol., 185:3-7, 1990.
Guo et al., Nature, 360:364-366, 1992.
Gupta et al., Biomaterials, 26:3995-4021, 2005.
Hanibuchi et al., Int. J. Cancer, 78(4):480-485, 1998.
Heemskerk et al. Hum Gene Ther. 19:496-510, 2008.
Hellstrand et al., Acta Oncologica, 37(4):347-353, 1998.
Hollander, Front. Immun., 3:3, 2012.
Hui and Hashimoto, Infection Immun., 66(11):5329-5336, 1998.
Hurwitz et al. (1998) Proc Natl Acad Sci USA 95(17): 10067-10071
International Patent Publication No. WO 00/37504
International Patent Publication No. WO 01/14424
International Patent Publication No. WO 98/42752
International Patent Publication No. WO 99/60120
International Patent Publication No. WO1995001994
International Patent Publication No. WO1998042752
International Patent Publication No. WO2000037504
International Patent Publication No. WO200014257
International Patent Publication No. WO2001014424
International Patent Publication No. WO2006/121168
International Patent Publication No. WO2007/103009
International Patent Publication No. WO2009/101611
International Patent Publication No. WO2010/027827
International Patent Publication No. WO2011/066342
International Patent Publication No. WO2012/129514
International Patent Publication No. WO2013/071154
International Patent Publication No. WO2013/123061
International Patent Publication No. WO2013/166321
International Patent Publication No. WO2013126726
International Patent Publication No. WO2014/055668
International Patent Publication No. WO2014031687
International Patent Publication No. WO2015016718
Janeway et al, Immunobiology: The Immune System in Health and Disease, 3^{rd} Ed., Current Biology Publications, p. 433, 1997.
Johnson et al. Blood 114:535-46, 2009.
Jores et al., PNAS U.S.A. 87:9138, 1990.
Kabat et al., “Sequences of Proteins of Immunological Interest, US Dept. Health and Human
Services, Public Health Service National Institutes of Health, 5^{th} ed, 1991.
Leal, M., Ann N Y Acad Sci 1321, 41-54, 2014.
Lefranc et al., Dev. Comp. Immunol. 27:55, 2003.
Li, Nat Biotechnol. 23:349-354, 2005.
Lin et al., J. Biol. Chem., 270:4255-14258, 1995.
Melief and van der Burg, Nat. Rev. Cancer, 8:351-360, 2008.
Mellman et al., Nature 480:480-489, 2011.
Mokyr et al. Cancer Res 58:5301-5304, 1998.
Moorthy et al., PLoS Med., 1(2):e33, 2004.
Neelapu et al., Blood, 15:109(12):5160-5163, 2007.
Pardoll, Nature Rev Cancer 12:252-264, 2012.
Parkhurst et al. Clin Cancer Res. 15: 169-180, 2009.
Popescu et al. Blood, 15:109(12):5407-5410, 2007.
Qin et al., Proc. Natl. Acad. Sci. USA, 95(24):14411-14416, 1998.
Quintarelli et al., Blood, 117:3353-3362, 2011.
Rojas et al., J. Biol. Chem., 271:27456-27461, 1996.
Rojas et al., Proc. West. Pharmacol. Soc., 41:55-56, 1998.
Sadelain et al., Cancer Discov. 3(4): 388-398, 2013.
Sambrook et al., Molecular Cloning: A Laboratory Manual, 3^{rd} ed., Cold Spring Harbor Press, 2001.
Samino et al., J. Biol. Chem., 281:6358-6365, 2006.
Schwarze et al., Trends in Cell Biol., 10:290-295, 2000.
Stryhn et al., Eur. J. Immunol., 30:3089-3099, 2000.
Terakura et al. Blood. 1:72-82, 2012.
Turtle et al., Curr. Opin. Immunol., 24(5): 633-39, 2012.
U.S. Pat. No. 4,373,071
U.S. Pat. No. 4,401,796
U.S. Pat. No. 4,458,066
U.S. Pat. No. 4,598,049
U.S. Pat. No. 4,870,287
U.S. Pat. No. 5,739,169
U.S. Pat. No. 5,760,395
U.S. Pat. No. 5,801,005
U.S. Pat. No. 5,824,311
U.S. Pat. No. 5,830,880
U.S. Pat. No. 5,844,905
U.S. Pat. No. 5,846,945
U.S. Pat. No. 5,885,796
U.S. Pat. No. 6,207,156
U.S. Pat. No. 6,225,042
U.S. Pat. No. 6,355,479
U.S. Pat. No. 6,362,001
U.S. Pat. No. 6,410,319
U.S. Pat. No. 6,451,995
U.S. Pat. No. 6,790,662
U.S. Pat. No. 7,070,995
U.S. Pat. No. 7,265,209
U.S. Pat. No. 7,354,762
U.S. Pat. No. 7,446,179
U.S. Pat. No. 7,446,190
U.S. Pat. No. 7,446,191
U.S. Pat. No. 7,666,604
U.S. Pat. No. 8,008,449
U.S. Pat. No. 8,017,114
U.S. Pat. No. 8,119,129
U.S. Pat. No. 8,252,592
U.S. Pat. No. 8,324,353
U.S. Pat. No. 8,329,867
U.S. Pat. No. 8,339,645
U.S. Pat. No. 8,354,509
U.S. Pat. No. 8,398,282
U.S. Pat. No. 8,479,118
U.S. Pat. No. 8,735,553
U.S. Patent Publication No. 2002/131960
U.S. Patent Publication No. 2005/0260186
U.S. Patent Publication No. 2006/0104968
U.S. Patent Publication No. 2009/0004142
U.S. Patent Publication No. 2009/0017000
U.S. Patent Publication No. 2011/0008369
U.S. Patent Publication No. 2013/0149337
U.S. Patent Publication No. 2013/287748
U.S. Patent Publication No. 2014/022021
U.S. Patent Publication No. 2014/0294898
Varela-Rohena et al. Nat Med. 14: 1390-1395, 2008.
Wang and Wang, Nat. Biotechnol., 20:149-154, 2002.
Wang et al. J Immunother 35(9):689-701, 2012.
Wu et al., Cancer, 18(2): 160-75, 2012.
Yee et al. Immunological reviews 257: 250-263. 2014.
Yee et al., J. Immunol. Methods, 261(1-2):1-20, 2002.
Young et al., J Exp. Med., 183:-11, 1996.
Zwaveling et al., J Immunol., 169:350-358, 2002.

What is claimed is:

1. An isolated VCX/Y peptide comprising
i) the amino acid sequence of SEQ ID NO: 1 (ASGPPAKAK), SEQ ID NO: 2 (SSQPSPSGPK), SEQ ID NO: 3 (SSQPSPSDPK), SEQ ID NO: 8 (RASGPPAKA), SEQ ID NO: 9 (AKAKETGKR), SEQ ID NO: 10 (KGAATKMAA), or SEQ ID NO: 11 (GAATKMAAV), or
ii) an amino acid sequence having no more than 1 amino acid substitution or deletion relative to SEQ ID NO:1, SEQ ID NO: 2, SEQ ID NO: 3, SEQ ID NO: 8, SEQ ID NO: 9, SEQ ID NO: 10, or SEQ ID NO: 11
wherein the peptide induces cytotoxic T lymphocytes (CTLs) and selectively binds to HLA.

2. The peptide of claim 1, wherein the isolated VCX/Y peptide is 35 amino acids in length or less.

3. The peptide of claim 1, wherein the peptide comprises:
i) SEQ ID NO: 1 and SEQ ID NO: 2 or
ii) SEQ ID NO: 1 and SEQ ID NO: 3 or
iii) an amino acid sequence having no more than 1 amino acid substitution or deletion relative to SEQ ID NO: 1 and an amino acid sequence having no more than 1 amino acid substitution or deletion relative to SEQ ID NO: 2 or
iv) or an amino acid sequence having no more than 1 amino acid substitution or deletion relative to SEQ ID NO: 1 and an amino acid sequence having no more than 1 amino acid substitution or deletion relative to SEQ ID NO: 3.

4. The peptide of claim 3, wherein the peptide is capable of inducing cytotoxic T lymphocytes (CTLs) and selectively binds to HLA-A11.

5. The peptide of claim 3, wherein the peptide is 32 amino acids or less in length and comprises the amino acid sequence of SEQ ID NO: 1 and SEQ ID NO: 2.

6. The peptide of claim 3, wherein the peptide is 32 amino acids or less in length and comprises the amino acid sequence of SEQ ID NO: 1 and SEQ ID NO: 3.

7. The peptide of claim 3, wherein the peptide comprises the amino acid sequence of SEQ ID NO: 4 (ASGPPAKAKETGKRKSSSQPSPSGPK) or SEQ ID NO: 5 (ASGPPAKAKETGKRKSSSQPSPSDPK).

8. The peptide of claim 3, wherein the peptide comprises the amino acid sequence of SEQ ID NO: 6 (MSPKPRASGPPAKAKETGKRKSSSQPSPSGPK) or SEQ ID NO: 7 (MSPKPRASGPPAKAKETGKRKSSSQPSPSDPK).

9. The peptide of claim 3, wherein the peptide comprises an amino acid sequence at least 90% identical to any one of SEQ ID NOs: 4-7.

10. The peptide of claim 9, wherein the peptide comprises an amino acid sequence at least 95% identical to any one of SEQ ID NOs: 4-7.

11. The peptide of claim 1, wherein the peptide is 32 amino acids or less in length and comprises the amino acid sequence of SEQ ID NO: 8, SEQ ID NO: 9, SEQ ID NO: 10, SEQ ID NO: 11 or an amino acid sequence having no more than 1 amino acid substitution or deletion relative to SEQ ID NO: 8, SEQ ID NO: 9, SEQ ID NO: 10, or SEQ ID NO: 11.

12. The peptide of claim 11, wherein the peptide is capable of inducing cytotoxic T lymphocytes (CTLs) and selectively binds to HLA-DR or HLA-DQ.

13. The peptide of claim 11, wherein the peptide is 26 amino acids or less in length and comprises the amino acid sequence of SEQ ID NO: 8.

14. The peptide of claim 13, wherein the peptide comprises the amino acid sequence of SEQ ID NO: 12 or an amino acid sequence at least 90% identical to SEQ ID NO: 12.

15. The peptide of claim 11, wherein the peptide wherein the peptide is 26 amino acids or less in length and comprises the amino acid sequence of SEQ ID NO: 9.

16. The peptide of claim 15, wherein the peptide comprises the amino acid sequence of SEQ ID NO: 13-14 or an amino acid sequence at least 90% identical to SEQ ID NO: 13-14.

17. The peptide of claim 11, wherein the peptide wherein the peptide is 26 amino acids or less in length and comprises the amino acid sequence of SEQ ID NO: 10.

18. The peptide of claim 17, wherein the peptide comprises the amino acid sequence of SEQ ID NO: 15 or an amino acid sequence at least 90% identical to SEQ ID NO: 15.

19. The peptide of claim 11, wherein the peptide is 26 amino acids or less in length and comprises the amino acid sequence of SEQ ID NO: 11.

20. The peptide of claim 19, wherein the peptide comprises the amino acid sequence of SEQ ID NO: 15 or an amino acid sequence at least 90% identical to SEQ ID NO: 15.

21. The peptide of claim 1, wherein the peptide is 30 amino acids in length or less.

22. The peptide of claim 21, wherein the peptide is 25 amino acids in length or less.

23. The peptide of claim 22, wherein the peptide is 20 amino acids in length or less.

24. The peptide of claim 21, wherein the peptide is 15 amino acids in length or less.

25. The peptide of claim 1, wherein the peptide consists of any one of SEQ ID NOs: 1-15.

26. A protein complex comprising a peptide according to any one of claims 1-25 in complex with HLA.

27. The complex of claim 26, wherein the HLA is a HLA-A11.

28. The complex of claim 26, wherein the HLA is a HLA-DR or HLA-DQ.

29. A pharmaceutical composition comprising the peptide of any one of claims 1-25 and a pharmaceutical carrier.

30. The pharmaceutical composition of claim 29, wherein the pharmaceutical carrier is a buffer salt solution.

31. The pharmaceutical composition of claim 29, wherein the pharmaceutical carrier is phosphate buffered saline, Ringer's lactate or a saline solution.

32. The composition of claim 29, wherein the pharmaceutical composition is formulated for parenteral administration, intravenous injection, intramuscular injection, inhalation, or subcutaneous injection.

33. The composition of claim 29, wherein the peptide is complexed with or is contained within a liposome, lipid-containing nanoparticle, or in a lipid-based carrier.

34. The composition of claim 29, wherein the pharmaceutical preparation is formulated for injection or inhalation as a nasal spray.

35. The composition of claim 32, further comprising an adjuvant component.

36. An isolated nucleic acid encoding the peptide of any one of claims 1-25.

37. A vector comprising a contiguous sequence of the nucleic acid of claim 36.

38. A host cell comprising the peptide of any one of claims 1-25 or the vector of claim 37.

39. The cell of claim 38, wherein the cell is not a cancer cell.

40. The cell of claim 38, wherein the cell is an antigen presenting cell.

41. The cell of claim 40, wherein the antigen presenting cell expresses the VCX/Y peptide of claim 1 on its surface.

42. The cell of claim 40 or 41, wherein the antigen presenting cell is a dendritic cell.

43. A method of promoting an immune response in a subject, comprising administering an effective amount of the peptide of any one of claims 1-25 to the subject, wherein the peptide induces VCX/Y-specific T cells in the subject.

44. The method of claim 43, wherein the subject is diagnosed with cancer.

45. The method of claim 44, wherein the cancer is testicular cancer, thymoma, bladder cancer, uterine carcinoma, melanoma, sarcoma, cervix cancer, or head and neck cancer.

46. The method of claim 43, wherein the subject is a human.

47. The method of claim 43, further comprising administering at least a second anti-cancer therapy.

48. The method of claim 47, wherein the second anti-cancer therapy is selected from the group consisting of a chemotherapy, a radiotherapy, an immunotherapy, or a surgery.

49. The method of claim 48, wherein the immunotherapy comprises at least one immune checkpoint inhibitor.

50. The method of claim 49, wherein the immune checkpoint inhibitor is an anti-PD1, anti-PDL1 or anti-CTLA-4 monoclonal antibody.

51. The method of claim 49, wherein the immunotherapy is a combination of immune checkpoint inhibitors.

52. The method of claim 51, wherein the combination of immune checkpoint inhibitors is anti-PD1 and anti-CTLA-4 monoclonal antibodies.

53. The method of claim 51, wherein the combination of immune checkpoint inhibitors is anti-PDL1 and anti-CTLA-4 monoclonal antibodies.

54. The method of any of claims 50-52, wherein the PD-1 monoclonal antibody is selected from the group consisting of nivolumab, pembrolizumab and cemiplimab.

55. The method of any of claim 50, 51 or 53, wherein the PD-L1 monoclonal antibody is selected from the group consisting of atezolizumab, avelumab and durvalumab.

56. The method of any of claims 50-53, wherein the CTLA-4 monoclonal antibody is ipilimumab.

57. A VCX-Y-specific immune effector cell that is specific for a VCX/Y peptide of any one of claims 1-25.

58. The cell of claim 57, wherein the immune effector cells are T cells, peripheral blood lymphocytes, NK cells, invariant NK cells, NKT cells.

59. A method of producing VCX/Y-specific immune effector cells comprising:
(a) obtaining a starting population of immune effector cells; and
(b) contacting the starting population of immune effector cells with the VCX/Y peptide of any one of claims 1-25, thereby generating VCX/Y-specific immune effector cells.

60. The method of claim 1, wherein the method is carried out in vitro.

61. The method of claim 59, wherein contacting is further defined as co-culturing the starting population of immune effector cells with antigen presenting cells (APCs), wherein the APCs present the VCX/Y peptide of claim 1 on their surface.

62. The method of claim 61, wherein the APCs are dendritic cells.

63. The method of claim 59, wherein the immune effector cells are T cells, peripheral blood lymphocytes, NK cells, invariant NK cells, NKT cells.

64. The method of claim 59, wherein the immune effector cells have been differentiated from mesenchymal stem cell (MSC) or induced pluripotent stem (iPS) cells.

65. The method of claim 63, wherein the T cells are CD8⁺ T cells, CD4⁺ T cells, or γδ T cells.

66. The method of claim 63, wherein the T cells are cytotoxic T lymphocytes (CTLs).

67. The method of claim 59, wherein obtaining comprises isolating the starting population of immune effector cells from peripheral blood mononuclear cells (PBMCs).

68. The method of any of claims 59-67, wherein the starting population of immune effector cells is obtained from a subject.

69. The method of claim 68, wherein the subject is a human.

70. The method of claim 69, wherein the subject has cancer.

71. The method of claim 62, wherein the method further comprises introducing the VCX/Y peptides or a nucleic acid encoding the VCX/Y peptide into the dendritic cells prior to the co-culturing.

72. The method of claim 71, where the peptide or nucleic acids encoding the peptide are introduced by electroporation.

73. The method of claim 71, wherein the peptide or nucleic acids encoding the peptide are introduced by adding the peptide or nucleic acid encoding the peptide to the dendritic cell culture media.

74. The method of claim 71, wherein the immune effector cells are co-cultured with a second population of dendritic cells into which the peptide or the nucleic acid encoding the peptide has been introduced.

75. The method of claim 71, wherein a population of CD8-positive and VCX/Y peptide WIC tetramer-positive T cells are purified from the immune effector cells following the co-culturing.

76. The method of claim 75, wherein a clonal population of VCX/Y-specific immune effector cells are generated by limiting or serial dilution followed by expansion of individual clones by a rapid expansion protocol.

77. The method of claim 76, wherein the method further comprises cloning of a T cell receptor (TCR) from the clonal population of VCX/Y-specific immune effector cells.

78. The method of claim 77, wherein cloning of the TCR is cloning of a TCR alpha and a beta chain.

79. The method of claim 77 or claim 78, wherein the TCR is cloned using a 5′-Rapid amplification of cDNA ends (RACE) method.

80. The method of claim 79, wherein the cloned TCR is subcloned into an expression vector.

81. The method of claim 80, wherein the expression vector is a retroviral or lentiviral vector.

82. The method of claim 81, where a host cell is transduced with the expression vector to generate an engineered cell that expresses the TCR.

83. The method of claim 82, wherein the host cell is an immune cell.

84. The method of 77, wherein the immune cell is a T cell and the engineered cell is an engineered T cell.

85. The method of claim 84, wherein the T cell is a CD8⁺ T cell, CD4+ T cell, or γδ T cell and the engineered cell is an engineered T cell.

86. The method of claim 85, wherein the starting population of immune effector cells is obtained from a subject with cancer and the host cell is allogeneic or autologous to the subject.

87. The method of claim 84 or 85, wherein a population of CD8-positive and VCX/Y peptide WIC tetramer-positive engineered T cells are purified from the transduced host cells.

88. The method of claim 75, wherein a clonal population of VCX/Y-specific engineered T cells are generated by limiting or serial dilution followed by expansion of individual clones by a rapid expansion protocol.

89. An VCX/Y-specific engineered T cell produced according to any one of the methods of claims 82-88.

90. A VCX/Y-specific T cell produced according to any one of the methods of claims 59-70.

91. A pharmaceutical composition comprising the VCX/Y-specific T cells produced according to any one of the methods of claims 59-70.

92. A method of treating cancer in a subject comprising administering an effective amount of the VCXIY-specific T cells of claim 89 or 90 to the subject.

93. A composition comprising an effective amount of the VCX/Y-specific T cells of claim 89, 90 or 148 for the treatment of cancer in a subject.

94. The method of claim 92, wherein the cancer is thymoma, bladder cancer, uterine carcinoma, melanoma, sarcoma, cervix cancer, or head and neck cancer.

95. The method of claim 92, wherein the subject is a human.

96. The method of claim 92, wherein the VCX/Y-specific T cells are autologous or allogeneic.

97. The method of claim 92, further comprising lymphodepletion of the subject prior to administration of the VCX/Y-specific T cells.

98. The method of claim 97, wherein lymphodepletion comprises administration of cyclophosphamide and/or fludarabine.

99. The method of claim 92, further comprising administering at least a second therapeutic agent.

100. The method of claim 99, wherein the at least a second therapeutic agent comprises chemotherapy, immunotherapy, surgery, radiotherapy, or biotherapy.

101. The method of claim 100, wherein the immunotherapy comprises at least one immune checkpoint inhibitor.

102. The method of claim 101, wherein the immune checkpoint inhibitor is an anti-PD1, anti-PDL1 or anti-CTLA-4 monoclonal antibody.

103. The method of claim 101, wherein the immunotherapy is a combination of immune checkpoint inhibitors.

104. The method of claim 103, wherein the combination of immune checkpoint inhibitors is anti-PD1 and anti-CTLA-4 monoclonal antibodies.

105. The method of claim 103, wherein the combination of immune checkpoint inhibitors is anti-PDL1 and anti-CTLA-4 monoclonal antibodies.

106. The method of any of claims 102-104, wherein the PD-1 monoclonal antibody is selected from the group consisting of nivolumab, pembrolizumab and cemiplimab.

107. The method of any of claim 102, 103 or 105, wherein the PD-L1 monoclonal antibody is selected from the group consisting of atezolizumab, avelumab and durvalumab.

108. The method of any of claims 102-105, wherein the CTLA-4 monoclonal antibody is ipilimumab.

109. The method of claim 99, wherein the VCX/Y-specific T cells and/or the at least a second therapeutic agent are administered intravenously, intraperitoneally, intratracheally, intratumorally, intramuscularly, endoscopically, intralesionally, percutaneously, subcutaneously, regionally, or by direct injection or perfusion.

110. The method of claim 92, wherein the subject is determined to have cancer cells which express a protein of the VCX/Y family.

111. The method of claim 110, wherein the protein is VCX3B or VCY.

112. A method of cloning a VCX/Y T cell receptor (TCR), the method comprising
(a) obtaining a starting population of immune effector cells;
(b) contacting the starting population of immune effector cells with the VCX/Y peptide of any one of claims 1-25, thereby generating VCX/Y-specific immune effector cells;
(c) purifying immune effector cells specific to the VCX/Y peptide,
(d) isolating a TCR sequence from the purified immune effector cells.

113. The method of claim 112, wherein contacting is further defined as co-culturing the starting population of immune effector cells with antigen presenting cells (APCs), wherein the APCs present the VCX/Y peptide of claim 1 on their surface.

114. The method of claim 113, wherein the APCs are dendritic cells.

115. The method of claim 112, wherein the immune effector cells are T cells, peripheral blood lymphocytes, NK cells, invariant NK cells, NKT cells.

116. The method of claim 112, wherein the immune effector cells have been differentiated from mesenchymal stem cell (MSC) or induced pluripotent stem (iPS) cells.

117. The method of claim 115, wherein the T cells are CD8⁺ T cells, CD4⁺ T cells, or γδ T cells.

118. The method of claim 115, wherein the T cells are cytotoxic T lymphocytes (CTLs).

119. The method of claim 112, wherein obtaining comprises isolating the starting population of immune effector cells from peripheral blood mononuclear cells (PBMCs).

120. The method of any of claims 112-119, wherein the starting population of immune effector cells is obtained from a subject.

121. The method of claim 120, wherein the subject is a human.

122. The method of claim 121, wherein the subject has cancer.

123. The method of claim 114, wherein the method further comprises introducing the VCX/Y peptide or a nucleic acid encoding the VCX/Y peptide into the dendritic cells prior to the co-culturing.

124. The method of claim 123, where the peptide or nucleic acid encoding the peptide are introduced by electroporation.

125. The method of claim 123, wherein the peptide or nucleic acid encoding the peptide are introduced by adding the peptide or nucleic acid encoding the peptide to the media of the dendritic cells.

126. The method of claim 123, wherein the immune effector cells are co-cultured with a second population of dendritic cells into which the VCX/Y peptide or a nucleic acid encoding the VCX/Y peptide has been introduced.

127. The method of claim 123, wherein purifying is defined as purifying a population of CD8-positive and VCX/Y peptide WIC tetramer-positive T cells from the immune effector cells following the co-culturing.

128. The method of claim 127, wherein the population of CD8-positive and VCX/Y peptide WIC tetramer-positive T cells are purified by fluorescence activated cell sorting (FACS).

129. The method of claim 128, wherein purifying further comprises generation of a clonal population of VCX/Y-specific immune effector cells by limiting or serial dilution of sorted cells followed by expansion of individual clones by a rapid expansion protocol.

130. The method of claim 129, wherein isolating is defined as cloning of a T cell receptor (TCR) from the clonal population of VCX/Y-specific immune effector cells.

131. The method of claim 130, herein cloning of the TCR is cloning of a TCR alpha and a beta chain.

132. The method of claim 131, wherein the TCR alpha and beta chains are cloned using a 5′-Rapid amplification of cDNA ends (RACE) method.

133. The method of claim 132, wherein the cloned TCR is subcloned into an expression vector.

134. The method of claim 133, wherein the expression vector comprises a linker domain between the TCR alpha sequence and TCR beta sequence.

135. The method of claim 134, wherein the linker domain comprises a sequence encoding one or more peptide cleavage sites.

136. The method of claim 135, wherein the one or more cleavage sites are a Furin cleavage site and/or a P2A cleavage site.

137. The method of claim 136, wherein the one or more cleavage sites are separated by a spacer.

138. The method of claim 137, wherein the spacer is SGSG or GSG.

139. The method of claim 138, wherein the TCR alpha sequence and TCR beta sequence are linked by an IRES sequence.

140. The method of any of claims 133-139, wherein the expression vector is a retroviral or lentiviral vector.

141. The method of claim 140, where a host cell is transduced with the expression vector to generate an engineered cell that expresses the TCR alpha and beta chains.

142. The method of claim 141, wherein the host cell is an immune cell.

143. The method of claim 142, wherein the immune cell is a T cell and the engineered cell is an engineered T cell.

144. The method of claim 143, wherein the T cell is a CD8⁺ T cell, CD4+ T cell, or γδ T cell and the engineered cell is an engineered T cell.

145. The method of claim 144, wherein a population of CD8-positive and VCX/Y peptide MHC tetramer-positive engineered T cells are purified from the transduced host cells.

146. The method of claim 145, wherein a clonal population of VCX/Y-specific engineered cells are generated by limiting or serial dilution after purification followed by expansion of individual engineered T cell clones by a rapid expansion protocol.

147. An engineered T cell that recognizes a VCX/Y peptide of any one of SEQ ID NOs:1-15 or a peptide of any one of claims 1-24.

148. A VCX/Y-specific engineered T cell expressing a TCR produced according to any one of the methods of claims 112-146.

149. A method of inducing an immune response in a subject comprising administering an effective amount of the T cells of claim 147 to the subject.
